SEQUENCE LISTING (59 sequences)

SEQ ID NO: 1 - DNA, 52 bp, Homo sapiens
tgagtacacagcgctcccgtcgcggcscccttgatgcaggaccctccatcgc

SEQ ID NO: 2 - DNA, 52 bp, Homo sapiens
acatgtaccttcaaggaactggtataygaaacagtgagagtgcccggctgtg

SEQ ID NO: 3 - DNA, 52 bp, Homo sapiens
cctgggacaaggacactgcttcacccrggtctgagaccgcagccccgagtcc

SEQ ID NO: 4 - DNA, 52 bp, Homo sapiens
caccccatcaatgccatcctggctgtsgagaaggagggctgcccagtgtgca

SEQ ID NO: 5 - DNA, 52 bp, Homo sapiens
ctacagaaccgatggcctgcctctaayggctggctcattggtacagtgagga

SEQ ID NO: 6 - DNA, 52 bp, Homo sapiens
ctgctcttcagctcccagagtcaccartggttccacttacatacttgtccct

SEQ ID NO: 7 - DNA, 52 bp, Homo sapiens
attcattcattcaaacctatacttacygaatgctcactaaatgccgggggtt

SEQ ID NO: 8 - DNA, 18 bp, Artificial
  Synthetic primer for amplifying APOE exon 4
ggcacggctgtccaagga

SEQ ID NO: 9 - DNA, 18 bp, Artificial
  Synthetic primer for amplifying APOE exon 4
ctggcggatggcgctgag

SEQ ID NO: 10 - DNA, 20 bp, Artificial
  Synthetic primer for amplifying LHbeta 5′
gttaccccaggcatcctatc

SEQ ID NO: 11 - DNA, 18 bp, Artificial
  Synthetic primer for amplifying LHbeta 5′
ccattccccaaccgcagg

SEQ ID NO: 12 - DNA, 21 bp, Artificial
  Synthetic primer for amplifying LHbeta 3′
ggtcctgaataggagatgcca

SEQ ID NO: 13 - DNA, 18 bp, Artificial
  Synthetic primer for amplifying LHbeta 3′
cggggtgtcagggctcca

SEQ ID NO: 14 - DNA, 19 bp, Artificial
  Synthetic primer for amplifying LHCGR exon 1
cactcagaggccgtccaag

SEQ ID NO: 15 - DNA, 20 bp, Artificial
  Synthetic primer for amplifying LHCGR exon 1
ggagggaaggtggcatagag

SEQ ID NO: 16 - DNA, 20 bp, Artificial
  Synthetic primer for amplifying LHCGR exon 10
acagtcaggtttagcctgaa

SEQ ID NO: 17 - DNA, 20 bp, Artificial
  Synthetic primer for amplifying LHCGR exon 10
cttctgagtttccttgcatg

SEQ ID NO: 18 - DNA, 22 bp, Artificial
  Synthetic primer for amplifying LHCGR exon 11
cagaaaatcccttacctcaagc

SEQ ID NO: 19 - DNA, 26 bp, Artificial
  Synthetic primer for amplifying LHCGR exon 11
ggtttaagaacaattcaataatgcag

SEQ ID NO: 20 - DNA, 24 bp, Artificial
  Synthetic primer for amplifying GnRH promoter
atagaggcagcattaggccttacc

SEQ ID NO: 21 - DNA, 24 bp, Artificial
  Synthetic primer for amplifying GnRH promoter
tggattcccttgaggaaaccagca

SEQ ID NO: 22 - DNA, 18 bp, Artificial
  Synthetic primer for amplifying GnRH 5′ untranslated region
gaagaatccaagagccag

SEQ ID NO: 23 - DNA, 24 bp, Artificial
  Synthetic primer for amplifying GnRH 5′ untranslated region
gcattactgctggctgaaccatct

SEQ ID NO: 24 - DNA, 24 bp, Artificial
  Synthetic primer for amplifying GnRH exon 1
tctgacttccatcttctgcagggt

SEQ ID NO: 25 - DNA, 24 bp, Artificial
  Synthetic primer for amplifying GnRH exon 1
agtgccttatctcacctggagcat

SEQ ID NO: 26 - DNA, 24 bp, Artificial
  Synthetic primer for amplifying GnRH exon 2
gcatttgacagcccaagggctaaa

SEQ ID NO: 27 - DNA, 23 bp, Artificial
  Synthetic primer for amplifying GnRH exon 2
aagtgccttatctacctggagca

SEQ ID NO: 28 - DNA, 23 bp, Artificial
  Synthetic primer for amplifying GnRHR exon 1A
acacaaggcttgaagctctgtcc

SEQ ID NO: 29 - DNA, 23 bp, Artificial
  Synthetic primer for amplifying GnRHR exon 1A
aagagcagcttcattcttgagag

SEQ ID NO: 30 - DNA, 21 bp, Artificial
  Synthetic primer for amplifying GnRHR exon 1B
acacagaagaaagagaaaggg

SEQ ID NO: 31 - DNA, 22 bp, Artificial
  Synthetic primer for amplifying GnRHR exon 1B
gctgttgcttttcaaagctagg

SEQ ID NO: 32 - DNA, 22 bp, Artificial
  Synthetic primer for amplifying GnRHR exon 1C
cttttctccatgtatgccccag

SEQ ID NO: 33 - DNA, 26 bp, Artificial
  Synthetic primer for amplifying GnRHR exon 1C
agaccttatatcaaatttagatagga

SEQ ID NO: 34 - DNA, 26 bp, Artificial
  Synthetic primer for amplifying GnRHR exon 2A
ctagcagagtaccaaagagaaaactt

SEQ ID NO: 35 - DNA, 22 bp, Artificial
  Synthetic primer for amplifying GnRHR exon 2A
agggatgatgaagaggcagctg

SEQ ID NO: 36 - DNA, 22 bp, Artificial
  Synthetic primer for amplifying GnRHR exon 2B
tagcagacagctctggacagac

SEQ ID NO: 37 - DNA, 21 bp, Artificial
  Synthetic primer for amplifying GnRHR exon 2B
aaactgcccacaaatgacact

SEQ ID NO: 38 - DNA, 24 bp, Artificial
  Synthetic primer for amplifying GnRHR exon 3A
cacctctcttttctctatccaaca

SEQ ID NO: 39 - DNA, 22 bp, Artificial
  Synthetic primer for amplifying GnRHR exon 3A
ccatagataagtgcatcaaagc

SEQ ID NO: 40 - DNA, 23 bp, Artificial
  Synthetic primer for amplifying GnRHR exon 3B
cctaggaatttggtattggtttg

SEQ ID NO: 41 - DNA, 24 bp, Artificial
  Synthetic primer for amplifying GnRHR exon 3B
acatttgtgttaatcattcccaga

SEQ ID NO: 42 - DNA, 27 bp, Artificial
  Synthetic primer for amplifying FSHbeta exon 3
tgttagagcaagcagtattcaatttct

SEQ ID NO: 43 - DNA, 26 bp, Artificial
  Synthetic primer for amplifying FSHbeta exon 3
gtatgtggcctgaaatgtccactgat

SEQ ID NO: 44 - DNA, 24 bp, Artificial
  Synthetic primer for amplifying FSHbeta 3′ untranslated region
agagcaaggtcagcatcttcagca

SEQ ID NO: 45 - DNA, 24 bp, Artificial
  Synthetic primer for amplifying FSHbeta 3′ untranslated region
ttgcaggagcctagtagcatgtga

SEQ ID NO: 46 - DNA, 24 bp, Artificial
  Synthetic primer for amplifying FSHR intron 1
tacagaaatgctggtgtggctcct

SEQ ID NO: 47 - DNA, 24 bp, Artificial
  Synthetic primer for amplifying FSHR intron 1
ccaaacaaagcacctgttgtcctc

SEQ ID NO: 48 - DNA, 24 bp, Artificial
  Synthetic primer for amplifying FSHR intron 8
tccctgtcatccaggaaccacttt

SEQ ID NO: 49 - DNA, 24 bp, Artificial
  Synthetic primer for amplifying FSHR intron 8
tctcagcggtgcctttcatgtagt

SEQ ID NO: 50 - DNA, 24 bp, Artificial
  Synthetic primer for amplifying FSHR exon 10, 5′ region
cccacattcaggttgtggcaagat

SEQ ID NO: 51 - DNA, 24 bp, Artificial
  Synthetic primer for amplifying FSHR exon 10, 5′ region
gctgctgatgccaaagatgggaaa

SEQ ID NO: 52 - DNA, 22 bp, Artificial
  Synthetic primer for amplifying FSHR exon 10, 3′ region
tgtcagtctacactctgacagc

SEQ ID NO: 53 - DNA, 24 bp, Artificial
  Synthetic primer for amplifying FSHR exon 10, 3′ region
gtgacatacccttcaaaggcaaga

SEQ ID NO: 54 - DNA, 24 bp, Artificial
  Synthetic primer for amplifying STAR intron 1
atggaaggcagatttctggaccct

SEQ ID NO: 55 - DNA, 24 bp, Artificial
  Synthetic primer for amplifying STAR intron 1
aagcctcagcacttaccgagtaga

SEQ ID NO: 56 - DNA, 24 bp, Artificial
  Synthetic primer for amplifying STAR exon 7
agctgattaatgggccctggaaga

SEQ ID NO: 57 - DNA, 24 bp, Artificial
  Synthetic primer for amplifying STAR exon 7
cccaatgtgtgtgtgtgtgtgtgt

SEQ ID NO: 58 - DNA, 24 bp, Artificial
  Synthetic primer for amplifying A2M exon 24
tggctgtggagagcagaatatggt

SEQ ID NO: 59 - DNA, 26 bp, Artificial
  Synthetic primer for amplifying A2M exon 24
ggaggttggagagtggatagtttcct

(19) United States
(12) Patent Application Publication
(10) Pub. No.: US 2017/0016072 A1
(43) Pub. Date: Jan. 19, 2017

(54) METHODS FOR ASSESSING RISK OF ALZHEIMER'S DISEASE IN A PATIENT

(71) Applicant: Wisconsin Alumni Research Foundation, Madison, WI (US)

(72) Inventor: Craig S. Atwood, Madison, WI (US)

(21) Appl. No.: 15/280,078

(22) Filed: Sep. 29, 2016

Related U.S. Application Data
(63) Continuation of application No. 13/691,048, filed on Nov. 30, 2012, now abandoned.
Application No. 13/691,048 is a continuation of application No. 13/205,289, filed on Aug. 8, 2011, now abandoned.
Application No. 13/205,289 is a continuation of application No. 12/510,607, filed on Jul. 28, 2009, now abandoned.
(60) Provisional application No. 61/084,377, filed on Jul. 29, 2008.

(51) Int. Cl. C12Q 1/68

(52) U.S. Cl. CPC C12Q 1/6883; C12Q 2600/172; C12Q 2600/156; C12Q 2600/16; C12Q 2600/118

(57) ABSTRACT

Disclosed are methods for diagnosis or prognosis of Alzheimer's disease in a patient. The methods may include assessing whether a patient has Alzheimer's disease or assessing a patient's risk for developing Alzheimer's disease. The methods typically include determining, either directly or indirectly, whether the patient has mutations, such as single nucleotide polymorphisms, in a plurality of genes that encode gene products that function in steroid biosynthesis.

### CROSS-REFERENCE TO RELATED APPLICATIONS

The present application claims the benefit under 35 U.S.C. §119(e) to U.S. Provisional Patent Application No. 61/084,377, filed on Jul. 29, 2008, the content of which is incorporated herein by reference in its entirety.

### BACKGROUND

The invention relates to methods for assessing risk in a patient for developing Alzheimer's disease. In particular, the methods related to detecting mutations in a plurality of genes present in a nucleic acid sample from a patient, where the genes encode gene products that function in the steroidogenic pathway (i.e., steroid biosynthesis and in particular neurosteroid biosynthesis). The detected mutations may include single nucleotide polymorphisms.

Alzheimer's disease (AD) is a progressive neurodegenerative disorder characterized by neuronal and synaptic loss, neurofibrillary tangles in neuronal cytoplasm, and deposition of β-amyloid (Aβ) in extracellular, neuritic plaques. To date, only four genes have been unambiguously associated with AD, of which only one, Apolipoprotein E (APOE), is associated with the common, late-onset form of AD [see Bertram L et al., Pharmacol Res 2004, 50(4):385-396]. The APOE4 allele (ε4) was first identified as a risk factor for late-onset AD in the early 1990s [see Corder E H et al., Science 1993, 261(5123):921-923; and Saunders A M et al., Neurology 1993, 43(8):1467-1472], and corroborated as such by a number of subsequent studies [see Farrer L A et al., Jama 1997, 278(16):1349-1356]. However, the risk for AD imparted by one or two ε4 alleles is only partially penetrant: ˜50% of AD patients do not carry an ε4 allele [see Roses A et al., Am J Hum Genet 1995, Suppl. 57:A202]. Application of quantitative genetics methodology in fact supports the presence of four (4) as yet unidentified AD-associated loci in the human genome, each expected to affect age of onset (AoO) as much or more than APOE [see Daw E W et al., Am J Hum Genet 2000, 66(1):196-204]. Additional genetic risk factors for AD, therefore, remain to be found. Yet, a majority of studies have failed to find any evidence for association of their genetic target(s) with AD (e.g., recently, Chapuis et al. [see Chapuis J et al., Neurobiol Aging 2006, 27(9):1212-1215] and [see Ozturk A et al., Neurosci Lett 2006, 406(3):265-269]), and large-scale meta-analyses, which combine the datasets of numerous studies, often negate or call into question any putative associations inferred from individual datasets [see Pritchard A et al., Neurosci Lett 2005, 382(3):221-226]).

The disproportionate number of women who suffer from AD has long suggested that an aspect of reproductive physiology lies at the origin of AD pathogenesis. Recently, this idea was supported by the discovery that polymorphisms of the estrogen receptors alpha and beta were associated with AD, further implicating estradiol signaling in the pathogenesis of AD [see Monastero R et al., J Alzheimers Dis 2006, 9(3):273-278; and Pirskanen M et al., Eur J Hum Genet 2005, 13(9):1000-1006]. Several converging lines of evidence make another member of the hypothalamic-pituitary-gonadal axis, luteinizing hormone (LH), a worthwhile candidate for genetic study: (1) LH is elevated in AD patients [see Bowen R L et al., J Neuroendocrinol 2000, 12(4):351-354; Short R A et al., Mayo Clin Proc 2001, 76(9):906-909; and Hogervorst E et al., Exp Gerontol 2004, 39(11-12):1633-1639]; (2) LH crosses the blood-brain barrier [see Lukacs H et al., Horm Behav 1995, 29(1):42-58]; (3) in the brain, LH/chorionic gonadotropin receptors (“LHCGR” or “LHR”) are most concentrated in the hippocampus [see Lei Z M et al., Mol Endocrinol 1994, 8(8):1111-1121]; (4) increased concentration of LH has been shown to increase Aβ secretion in a neuronal cell line while suppression of serum LH decreases brain Aβ in mice [see Bowen R L et al., J Biol Chem 2004, 279(19):20539-20545]; and, (5) reduced serum LH has been shown to decrease cognitive loss and Aβ deposition in AβPP transgenic mice [see Casadesus G et al., Biochem Biophys Acta 2006]. Interestingly, through its regulation of steroidogenic enzymes, LH mediates neurosteroid production from cholesterol [see Liu T et al., J Neurochem 2007, 100(5):1329-1339]; both animal and human clinical studies strongly support the crucial neuroprotective functions of steroids in the brain [see Weill-Engerer S et al., J Clin Endocrinol Metab 2002, 87(10:5138-5143; and see Simpkins J W et al., Cell Mol Life Sci 2005, 62(3):271-280]. Since APOE is a cholesterol transport protein [see Mahley R W et al., Science 1988, 240(4852):622-630] involved in the transport of cholesterol into neurons [see Andersen O M et al., Trends Neurosci 2006, 29(12):687-694] for neurosteroid synthesis, a functional link exists between APOE and LH signaling.

Numerous polymorphisms of LH beta-subunit (LHB) and LHR have been documented (for comprehensive reviews, see [Themmen A P N et al., Endocr Rev 2000, 21(5):551-583; and Huhtaniemi I T et al., Endocrine 2005, 26(3):207-217]). While the majority of mutations underlying these polymorphisms are associated with rare reproductive disorders, a few are relatively more common and worthy of exploring for their association with AD. Two non-synonymous single nucleotide polymorphisms (SNPs) in LHB are collectively referred to as variant LH (vLH) [see Furui K et al., J Clin Endocrinol Metab 1994, 78(1):107-113]. In a study of 40 Japanese women, vLH carriers exhibited greater LH secretion in response to GnRH stimulation [see Takahashi K et al., Eur J Endocrinol 2000, 143(3):375-381]. In breast cancer patients, an LQ-insert in exon 1 of LHR was associated with a significantly earlier age of onset and worse survival rate [see Takahashi K et al., Eur J Endocrinol 2000, 143(3):375-381]. Exon 10 of LHR is required for binding of LH [see Muller T et al., J Clin Endocrinol Metab 2003, 88(5):2242-2249] and is the location of 2 relatively common non-synonymous SNPs [see Richter-Unruh A et al., Clin Endocrinol (Oxf) 2002, 56(1):103-112]. The functional consequences of the mutations underlying other LHB and LHR polymorphisms are largely unknown.

Here, polymorphic sites of LH β-subunit (LHB) and LHR were studied, as well as gene-gene interactions between LHB, LHR, and APOE for association with AD. The present results suggest that a specific LHR allele modulates the risk of AD in individuals carrying an APOE ε4 allele. In addition to studying polymorphic sites of LH β-subunit (LHB) and LHR, polymorphic sites of other members of the steroidogenic pathway were studied here, including polymorphic sites of follicle stimulating hormone (FSH). The present results suggest that a specific FSH allele also significantly modulates the risk of AD in individuals carrying an APOE ε4 allele. All these results together may suggest that other members of the steroidogenic pathway modulate the risk of AD, particularly in those patients carrying an APOE ε4 allele.

### SUMMARY

Disclosed are methods and kits for diagnosis or prognosis of Alzheimer's disease (AD) in a patient. The methods may include assessing whether a patient has AD or assessing the likelihood of the patient developing AD. The methods typically include detecting mutations in a plurality of genes present in a nucleic acid sample from the patient, where the genes encode gene products that function in the steroidogenic pathway (i.e., steroid biosynthesis and in particular neurosteroid biosynthesis). The detected mutations may include single nucleotide polymorphisms.

In some embodiments, the methods may include detecting, either directly or indirectly, the presence or absence of specific single nucleotide polymorphisms (SNPs) in a plurality of genes of the steroidogenic pathway. (See FIG. 1 and Table 2 with respect to steroidogenic genes and dbSNP reference ID Nos.) Mutations in genes of the steroidogenic pathway may include but are not limited to single nucleotide polymorphisms in genes encoding follicle stimulating hormone (e.g., FSH1 and FSH2), follicle stimulating hormone receptor (e.g., FSHR1, FSHR2, FSHR3, FSHR4, FSHR5, FSHR6, FSHR7, FSHR8, FSHR9, FSHR10, FSHR11, FSHR12, and FSHR13), luteinizing hormone beta (e.g., LHB1, LHB2, LHB3, LHB4, LHB5, and LHB6), luteinizing hormone receptor (e.g., LHR1, LHR2, LHR3, LHR4, and LHR5), and gonadotropin-releasing hormone (e.g, Gpro and GX1). For example, the methods may include detecting, either directly or indirectly, the presence or absence of specific polymorphisms in the luteinizing hormone receptor gene such as rs4073366, a polymorphism in the follicle stimulating hormone gene such as rs6169, or preferably both polymorphisms. Genes of the steroidogenic pathway may function in the synthesis of steroids such as pregnenolone and progesterone, which are known to be neurosteroids (i.e., neuroactive).

In some embodiments, the methods include: (a) obtaining a nucleic acid sample from the patient; (b) identifying or detecting a nucleotide in the sample at a nucleotide position associated with single nucleotide polymorphism referred to by reference number rs4073366, reference number rs6169, or preferably both. The methods may also include determining the APOE genotype of the patient (e.g., determining whether the patient has an APOE2, APOE3, or APOE4 allele), either directly by detecting nucleic acid associated with the APOE2, APOE3, or APOE4 allele or indirectly by detecting the apolipoprotein E isoform (e.g., by an immunomethod or an immunoassay). The method further may include determining whether the patient is homozygous for the APOE2, APOE3, or APOE4 allele or whether the patient is heterozygous for the APOE2, APOE3, or APOE4 allele.

The methods may include identifying sex of the patient (i.e. either male or female) in assessing whether a patient has AD or whether a patient is likely to develop AD. In some embodiments, the methods may include determining whether the patient is female in assessing whether the patient is likely to develop AD.

The detected polymorphisms may include single nucleotide polymorphisms (SNPs). The SNPs may be detected by any suitable method, which may include, but is not limited to, nucleotide sequencing, probe hybridization, and primer specific PCR.

The detected SNP may be present in the luteinizing hormone receptor gene (LHR). For example, the detected SNP may be the SNP referred to by dbSNP reference No. rs4073366, which is a SNP C← →G transversion that is present in the first intron of the LHR gene. The disclosed methods directly may identify a C nucleotide at the position associated with the SNP rs4073366, thereby indicating that the patient has a C-allele, or the disclosed methods directly may identify a G nucleotide at the position associated with the SNP rs4073366, thereby indicating that the patient has a G-allele. In some embodiments, the disclosed methods directly may identify both a C at the position associated with single nucleotide polymorphism rs4073366, thereby indicating that the patient has a C-allele, and further directly may identify a G at the position associated with the single nucleotide polymorphism rs4073366, thereby indicating that the patient has a G-allele. The disclosed methods may determine whether the patient is homozygous or heterozygous for the rs4073366 SNP.

The detected SNP may be present in the follicle stimulating hormone gene (FSH). For example, the detected SNP may be the SNP referred to by dbSNP reference No. rs6169, which is a SNP C4T transition that is present in the third exon of the FSH gene. The disclosed methods directly may identify a C nucleotide at the position associated with the SNP rs6169, thereby indicating that the patient has a C-allele, or the disclosed methods may directly identify a T nucleotide at the position associated with the SNP rs6169, thereby indicating that the patient has a T-allele. In some embodiments, the disclosed methods directly may identify both a C at the position associated with single nucleotide polymorphism rs6169, thereby indicating that the patient has a C-allele, and further directly may identify a T at the position associated with the single nucleotide polymorphism rs6169, thereby indicating that the patient has a T-allele. The disclosed methods may determine whether the patient is homozygous or heterozygous for the rs6169 SNP.

The disclosed methods may be utilized to assess whether a patient has AD or whether a patient is likely to develop AD. In some embodiments, the methods may be utilized to assess whether a patient has late-onset AD or whether a patient is likely to develop late-onset AD, for example where the patient is sixty-five (65) years of age or older.

The disclosed methods may be utilized to assess whether a patient has an increased or decreased risk for developing AD. In some embodiments, the methods may be utilized to assess whether the patient has a greater than about 80% risk of developing late-onset AD (or greater than about 85%, 90%, 95%, or 99% risk of developing late-onset AD). The methods also may be utilized to assess whether the patient has a reduced risk for developing AD. In some embodiments, the methods may be utilized to assess whether the patient has less than about 20% risk of developing late-onset AD).

In some embodiments, the methods may be utilized to determine that a patient has an increased risk for developing AD. For example, the methods may be utilized to determine that a patient has an increased risk for developing AD (e.g., a risk greater than about 99%) where: (i) the patient has at least one APOE4 allele; (ii) the patient is female; and (iii) the patient is homozygous for the C-allele or the G-allele for rs4073366. The methods may be utilized to determine that a patient has an increased risk for developing AD (e.g., at risk greater than about 90%) where: (i) the patient has at least one APOE4 allele; (ii) the patient is female; and (iii) and the patient has at least one C-allele for rs6169. The methods also may be utilized to determine that a patient has an increased risk for developing AD (e.g., at risk greater than about 85%) where: (i) the patient has at least one APOE4 allele; (ii) the patient is homozygous for the C-allele or the G-allele for rs4073366; and (iii) the patient has at least one C-allele for rs6169.

In some embodiments, the methods include: (a) obtaining a nucleic acid sample from the patient; (b) identifying or detecting a nucleotide in the sample at a nucleotide position associated with single nucleotide polymorphism referred to by reference number rs4002462; or (c) identifying or detecting a nucleotide in the sample at a nucleotide position associated with single nucleotide polymorphism referred to by reference number rs974894; or (d) performing both steps (b) and (c). In further embodiments, the methods include (a) obtaining a nucleic acid sample from the patient; (b) identifying or detecting a nucleotide in the sample at a nucleotide position associated with single nucleotide polymorphism referred to by reference number rs6166; or (c) and identifying or detecting a nucleotide in the sample at a nucleotide position associated with single nucleotide polymorphism referred to by reference number rs6521; or (d) performing both steps (b) and (c). In even further embodiments, the methods include (a) obtaining a nucleic acid sample from the patient; (b) identifying or detecting a nucleotide in the sample at a nucleotide position associated with single nucleotide polymorphism referred to by reference number rs974894; or (c) identifying or detecting a nucleotide in the sample at a nucleotide position associated with single nucleotide polymorphism referred to by reference number Gpro; or (d) performing both steps (b) and (c).

Also contemplated are kits for performing the disclosed methods. A kit may include a plurality of reagents for determining, either directly or indirectly, whether a patient has a single nucleotide polymorphism selected from rs4073366 and rs6169. A kit may include a plurality of reagents for identifying or detecting a nucleotide in the sample at a nucleotide position associated with single nucleotide polymorphism selected from rs4073366 and rs6169. The kit further may include a plurality of reagents for detecting an APOE2, APOE3, or APOE4 allele (e.g., a plurality of reagents for detecting APOE2, APOE3, or APOE4 nucleic acid or a plurality of reagents for detecting apolipoprotein E isoform such as an anti-apolipoprotein E isoform antibody).

Also contemplated are oligonucleotide arrays for performing the methods disclosed herein. In some embodiments, the oligonucleotide arrays may comprise a plurality of oligonucleotides for detecting the rs4073366 SNP, the rs6169 SNP, and the APOE allele.

### BRIEF DESCRIPTION OF THE DRAWINGS

FIG. 1. Steroidogenic Pathway.

FIG. 2. Key to Recursive Partitioning analyses.

FIG. 3. Interaction between FSH1, LHR2, and gender.

FIG. 4. Modulation of ε4 associated risk by FSH1 genotype.

FIG. 5. Modulation of ε4 associated risk by LHR2 genotype.

FIG. 6. Modulation of ε4 associated risk by gender.

FIG. 7. Modulation of ε4-associated risk by gender and LHR2.

FIG. 8. Modulation of ε4-associated risk by gender and FSH1.

FIG. 9. Modulation of ε4-associated risk by FSH1 and LHR2.

FIG. 10. Gender dependent interaction between LHβ2 and FSHR2.

FIG. 11. Interaction between FSHR13 and LHB4.

FIG. 12. Interaction between FSHR2 and GPRO.

### DETAILED DESCRIPTION

The present invention is described herein using several definitions, as set forth below and throughout the application.

As used in this specification and the claims, the singular forms “a,” “an,” and “the” include plural forms unless the content clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.”

As used herein, the term “plurality” means “two or more.”

As used herein, the term “patient,” which may be used interchangeably with the terms “subject” or “individual,” refers to one who receives medical care, attention or treatment and may encompass a human patient. As used herein, the term “patient” is meant to encompass a person at risk for developing Alzheimer's disease (AD) or a person diagnosed with AD (e.g., a person who may be symptomatic for AD but who has not yet been diagnosed).

As used herein the terms “diagnose” or “diagnosis” or “diagnosing” refer to distinguishing or identifying a disease, syndrome or condition or distinguishing or identifying a person having or at risk for developing a particular disease, syndrome or condition. As used herein the terms “prognose” or “prognosis” or “prognosing” refer to predicting an outcome of a disease, syndrome or condition. The methods contemplated herein include diagnosing an AD in a patient. The methods contemplated herein also include determining a prognosis for a patient having AD.

The term “sample” or “patient sample” is meant to include biological samples such as tissues and bodily fluids. “Bodily fluids” may include, but are not limited to, blood, serum, plasma, saliva, cerebral spinal fluid, pleural fluid, tears, lactal duct fluid, lymph, sputum, and semen. A sample may include nucleic acid, protein, or both.

The term “nucleic acid” or “nucleic acid sequence” refers to an oligonucleotide, nucleotide or polynucleotide, and fragments or portions thereof, which may be single or double stranded, and represents the sense or antisense strand. A nucleic acid may include DNA or RNA, and may be of natural or synthetic origin. For example, a nucleic acid may include mRNA or cDNA. Nucleic acid may include nucleic acid that has been amplified (e.g., using polymerase chain reaction).

An “amino acid sequence” refers to a polymer of amino acids present in a polypeptide or protein.

As used herein, the term “assay” or “assaying” means qualitative or quantitative analysis or testing.

As used herein the term “sequencing,” as in determining the sequence of a polynucleotide, refers to methods that determine the base identity at multiple base positions or that determine the base identity at a single position.

The term “amplification” or “amplifying” refers to the production of additional copies of a nucleic acid sequence. Amplification is generally carried out using polymerase chain reaction (PCR) technologies known in the art.

The present methods and kits may utilize primers, probes, or both. The term “primer” refers to an oligonucleotide that hybridizes to a target nucleic acid and is capable of acting as a point of initiation of synthesis when placed under conditions in which primer extension is initiated (e.g., primer extension associated with an application such as PCR). For example, primers contemplated herein may hybridize to one or more polynucleotide sequences of SEQ ID NOs:1-7. Primers as contemplated herein may comprise one or more polynucleotide sequences of SEQ ID NOs:8-59. A “probe” refers to an oligonucleotide that interacts with a target nucleic acid via hybridization. A primer or probe may be fully complementary to a target nucleic acid sequence or partially complementary. The level of complementarity will depend on many factors based, in general, on the function of the primer or probe. For example, probes contemplated herein may hybridize to one or more polynucleotide sequences of SEQ ID NOs:1-7. Probes as contemplated herein may comprise one or more polynucleotide sequences of SEQ ID NOs:8-59. A primer or probes can be used, for example to detect the presence or absence of a mutation in a nucleic acid sequence by virtue of the sequence characteristics of the target. Primers and probes can be labeled (e.g., with a fluorophore, a radiolabel, an enzyme, a particulate label, or the like) or unlabeled, or modified in any of a number of ways well known in the art. A primer or probe may specifically hybridize to a target nucleic acid (e.g., hybridize under stringent conditions as discussed herein).

The term “oligonucleotide” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

The present methods may be performed to detect the presence or absence of the disclosed SNPs. Methods of determining the presence or absence of a SNP may include a variety of steps known in the art, including one or more of the following steps: reverse transcribing mRNA that comprises the SNP to cDNA, amplifying nucleic acid that comprises the SNP (e.g., amplifying genomic DNA that comprises the SNP), hybridizing a probe or a primer to nucleic acid that comprises the SNP (e.g., hybridizing a probe to mRNA, cDNA, or amplified genomic DNA that comprises the SNP), and sequencing nucleic acid that comprises the SNP (e.g., sequencing cDNA or amplified DNA that comprises the SNP).

The term “heterozygous” refers to having different alleles at one or more genetic loci in homologous chromosome segments. As used herein “heterozygous” may also refer to a sample, a cell, a cell population or a patient in which different alleles (e.g., SNPs) at one or more genetic loci may be detected. Heterozygous samples may also be determined via methods known in the art such as, for example, nucleic acid sequencing. For example, if a sequencing electropherogram shows two peaks at a single locus and both peaks are roughly the same size, the sample may be characterized as heterozygous. Or, if one peak is smaller than another, but is at least about 25% the size of the larger peak, the sample may be characterized as heterozygous. In some embodiments, the smaller peak is at least about 15% of the larger peak. In other embodiments, the smaller peak is at least about 10% of the larger peak. In other embodiments, the smaller peak is at least about 5% of the larger peak. In other embodiments, a minimal amount of the smaller peak is detected.

As used herein, the term “homozygous” refers to having identical alleles (e.g., SNPs) at one or more genetic loci in homologous chromosome segments. “Homozygous” may also refer to a sample, a cell, a cell population, or a patient in which the same alleles at one or more genetic loci may be detected. Homozygous samples may be determined via methods known in the art, such as, for example, nucleic acid sequencing. For example, if a sequencing electropherogram shows a single peak at a particular locus, the sample may be termed “homozygous” with respect to that locus.

As used herein, the term “specific hybridization” indicates that two nucleic acid sequences share a high degree of complementarity. Specific hybridization complexes form under stringent annealing conditions and remain hybridized after any subsequent washing steps. Stringent conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art.

As used herein, a “target nucleic acid” refers to a nucleic acid molecule containing a sequence that has at least partial complementarity with a probe oligonucleotide, a primer oligonucleotide, or both. A primer or probe may specifically hybridize to a target nucleic acid.

A “polymorphism” refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is the replacement of one nucleotide by another nucleotide at the polymorphic site. Deletion of a single nucleotide or insertion of a single nucleotide also gives rise to single nucleotide polymorphisms. “Single nucleotide polymorphism” preferably refers to a single nucleotide substitution. Typically, between different individuals, the polymorphic site can be occupied by two different nucleotides. An individual may be homozygous or heterozygous for the single nucleotide polymorphism. “Mutation” as utilized herein, is intended to encompass a single nucleotide substitution, which is observed to be a single nucleotide polymorphism.

An “oligonucleotide array” refers to a substrate comprising a plurality of oligonucleotide primers or probes. The arrays contemplated herein may be used to detect the SNPs disclosed herein (e.g., rs4073366 and rs6169), and further may be used to detect an APOE allele (e.g., an APOE2, APOE3, or APOE4 allele).

As used herein, the term “APOE” refers to the apolipoprotein E. The methods disclosed herein may include detecting one or more alleles of the APOE gene (e.g., the APOE2, APOE3, or APOE4 allele). The APOE ε4 allele, which was first identified as a risk factor for late-onset AD in the early 1990s and corroborated as such by a number of subsequent studies. (See Corder et al., Science 1993, 261(5123):921-923; Saunders et al., Neurology 1993, 43(8):1467-1472; and Farrer et al., JAMA 1997, 278(16):1349-1356, which are incorporated herein by reference). Exemplary sequences for the apolipoprotein precursor polypeptide and apolipoprotein mRNA have been published. (See GenBank accession numbers NP_00032 and NM_000041, which publications are incorporated herein by reference). Exemplary sequences for the APOE4 allele have been published. (See GenBank accession number M10065.1 and Paik et al., Proc. Natl. Acad. Sci. USA 82(10), 3445-3449 (1985), which publications are incorporated herein by reference). As contemplated herein, an APOE allele may be detected directly (e.g., using methods for detecting or analyzing nucleic acid) or indirectly (e.g., by detecting apolipoprotein E using immunomethods).

The present methods contemplate detecting a single nucleotide polymorphism (SNP) in the gene for the luteinizing hormone receptor (LHR) (which alternately may be referred to as the gene for the luteinizing hormone/choriogonadotropin receptor (LHCGR)). For example, the present methods may detect the SNP referred to by dbSNP reference ID No. rs4073366 in either one or both alleles of the patient. (See rs407336 SNP entry at the National Center for Biotechnology Information, which entry is incorporated herein by reference and refers to a C← →G transversion at the reference position in the first intron of the LHR gene. See also polymorphism in SEQ ID NO:1—TGAGTACACAGCGCTCCCGTCGCGGCSCCCTTGATGCAGGACCCTCCATCGC.) This polymorphism may be referred to herein as “the LHR2 polymorphism.” The present methods may detect a C-allele or a G-allele corresponding to the polymorphism (i.e., a C-nucleotide or a G-nucleotide at the position associated with the rs407336 SNP). The present methods may detect whether a patient is homozygous or heterozygous for a C-allele or G-allele (i.e., whether the patient is C/C, G/G, or C/G at the reference nucleotide position). The present methods may detect the polymorphism directly by analyzing nucleic acid having the polymorphic variant sequence, or indirectly by analyzing an isoform polypeptide expressed from the polymorphic variant sequence.

The present methods contemplate detecting a single nucleotide polymorphism (SNP) in the gene for the follicle-stimulating hormone (FSH). For example, the present methods may detect the SNP referred to by dbSNP reference ID No. rs6169. (See rs6169 SNP entry at the National Center for Biotechnology Information, which entry is incorporated herein by reference and refers to a C← →T transition at the reference position in the third exon of the FSH gene. See also polymorphism in SEQ ID NO:2—ACATGTACCTTCAAGGAACTGGTATAYGAAACAGTGAGAGTGCCCGGCTGTG.) This polymorphism may be referred to herein as “the FSH1 polymorphism.” The present methods may detect a C-allele or a T-allele corresponding to the polymorphism (i.e., a C-nucleotide or a T-nucleotide at the position associated with the rs6169 SNP). The present methods may detect whether a patient is homozygous or heterozygous for a C-allele or T-allele (i.e., whether the patient is C/C, T/T, or C/T at the reference nucleotide position). The present methods may detect the polymorphism directly by analyzing nucleic acid having the polymorphic variant sequence, or indirectly by analyzing an isoform polypeptide expressed from the polymorphic variant sequence.

The present methods contemplate detecting a single nucleotide polymorphism (SNP) in the gene for the luteinizing hormone beta (LHbeta). For example, the present methods may detect the SNP referred to by dbSNP reference ID No. rs4002462. (See rs4002462 SNP entry at the National Center for Biotechnology Information, which entry is incorporated herein by reference and refers to a C← →T transition at the reference position in the first intron of the LHbeta gene. See also polymorphism in SEQ ID NO:3—CCTGGGACAAGGACACTGCTTCACCCRGGTCTGAGACCGCAGCCCCGAGTCC.) This polymorphism may be referred to herein as “the LHB2 polymorphism.” The present methods may detect a C-allele or a T-allele corresponding to the polymorphism (i.e., a C-nucleotide or a T-nucleotide at the position associated with the rs4002462 SNP). The present methods may detect whether a patient is homozygous or heterozygous for a C-allele or T-allele (i.e., whether the patient is C/C, T/T, or C/T at the reference nucleotide position). For another example, the present methods may detect the SNP referred to by dbSNP reference ID No. rs6521. (See rs6521 SNP entry at the National Center for Biotechnology Information, which entry is incorporated herein by reference and refers to a G transversion at the reference position in the second exon of the LHbeta gene. See also polymorphism in SEQ ID NO:4—CACCCCATCAATGCCATCCTGGCTGTSGAGAAGGAGGGCTGCCCAGTGTGCA.) This polymorphism may be referred to herein as “the LHB4 polymorphism.” The present methods may detect a C-allele or a G-allele corresponding to the polymorphism (i.e., a C-nucleotide or a G-nucleotide at the position associated with the rs6521 SNP). The present methods may detect whether a patient is homozygous or heterozygous for a C-allele or G-allele (i.e., whether the patient is C/C, G/G, or C/G at the reference nucleotide position). The present methods may detect the polymorphism directly by analyzing nucleic acid having the polymorphic variant sequence, or indirectly by analyzing an isoform polypeptide expressed from the polymorphic variant sequence.

The present methods contemplate detecting a single nucleotide polymorphism (SNP) in the gene for follicle stimulating hormone receptor (FSHR). For example, the present methods may detect the SNP referred to by dbSNP reference ID No. rs974894. (See rs974894 SNP entry at the National Center for Biotechnology Information, which entry is incorporated herein by reference and refers to a C← →T transition at the reference position in the first intron of the FSHR gene. See also polymorphism in SEQ ID NO:5—CTACAGAACCGATGGCCTGCCTCTAAYGGCTGGCTCATTGGTACAGTGAGGA.) This polymorphism may be referred to herein as “the FSHR2 polymorphism.” The present methods may detect a C-allele or a T-allele corresponding to the polymorphism (i.e., a C-nucleotide or a T-nucleotide at the position associated with the rs974894 SNP). The present methods may detect whether a patient is homozygous or heterozygous for a C-allele or T-allele (i.e., whether the patient is C/C, T/T, or C/T at the reference nucleotide position). For another example, the present methods may detect the SNP referred to by dbSNP reference ID No. rs6166. (See rs6166 SNP entry at the National Center for Biotechnology Information, which entry is incorporated herein by reference and refers to a A← →G transition at the reference position in the tenth exon of the FSHR gene. See also polymorphism in SEQ ID NO:6—CTGCTCTTCAGCTCCCAGAGTCACCARTGGTTCCACTTACATACTTGTCCCT.) This polymorphism may be referred to herein as “the FSHR13 polymorphism.” The present methods may detect an A-allele or a G-allele corresponding to the polymorphism (i.e., a A-nucleotide or a G-nucleotide at the position associated with the rs6166 SNP). The present methods may detect whether a patient is homozygous or heterozygous for an A-allele or G-allele (i.e., whether the patient is A/A, G/G, or A/G at the reference nucleotide position). The present methods may detect the polymorphism directly by analyzing nucleic acid having the polymorphic variant sequence, or indirectly by analyzing an isoform polypeptide expressed from the polymorphic variant sequence.

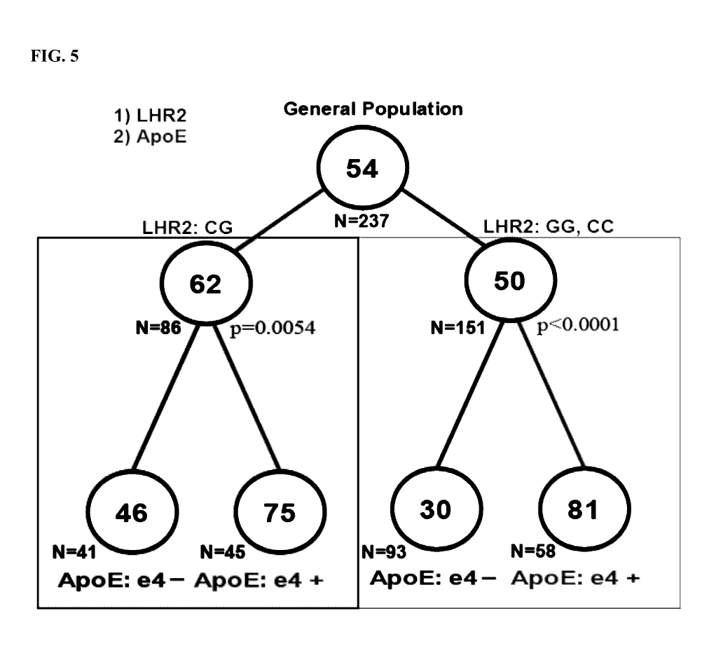

The present methods contemplate detecting a single nucleotide polymorphism (SNP) in the gene for gonadotropin-releasing hormone (GnRH). For example, the present methods may detect a novel SNP referred to as “Gpro” which is present in the promoter for the GNR gene at nucleotide position −1073 upstream of the transcriptional start site and refers to a C← →T transition. (See Wolfe et al., Molecular Endocrinology, 16(3):435-449 (2002), FIG. 5B for the sequence of the promoter region of GnRH, the content of which is incorporated by reference herein in its entirety. See also polymorphism in SEQ ID NO:7—ATTCATTCATTCAAACCTATACTTACYGAATGCTCACTAAATGCCGGGGGTT).

The methods may detect a C-allele or a T-allele corresponding to the polymorphism (i.e., a C-nucleotide or a T-nucleotide at the position associated with Gpro). The present methods may detect whether a patient is homozygous or heterozygous for a C-allele or T-allele (i.e., whether the patient is C/C, T/T, or C/T at the reference nucleotide position). The present methods may detect the polymorphism directly by analyzing nucleic acid having the polymorphic variant sequence, or indirectly by analyzing an isoform polypeptide expressed from the polymorphic variant sequence.

The present methods provide a screen for identifying people at a high risk of developing late onset AD (LO-AD). For example, methods provide a screen for identifying people at greater than 85%, 90%, 95%, or 99% risk for developing LO-AD. It is well accepted that women are at higher risk of developing LO-AD than men. This is due, in part, to women generally living longer than men. Other researchers have also reported that people expressing a specific isoform of apolipoprotein E, type 4, are more susceptible to the disease. A number of reproductive hormones appear to stimulate neuronal development. As such, the present inventor analyzed whether polymorphisms present in the cholesterol-sex steroid pathway in 250 LO-AD patients were associated with risk for developing AD. Two polymorphs, one in the gene encoding the luteinizing hormone receptor (LHR) and one encoding follicle stimulating hormone (FSH), were associated with an increased risk of developing the disease.

Individuals who are female, possess an ApoE4 allele and the rs4073366 LHR intron 1 SNP (i.e., “the LHR2 polymorphism”) are at 100% risk of developing AD. Individuals who are female, possess the ApoE4 allele and the rs6169 exon 3 synonymous SNP (i.e. “the FSH1 polymorphism”) are at a 90% risk of developing AD. Individuals who possess an Apoe4 allele and both LHR2 and FSH1 polymorphisms are at an 87% risk of developing AD. Therefore, a test is disclosed which provides a means of identifying people with at least an 87% increased risk of developing AD. The health of these identified patients may be better monitored early which hopefully will prolong a high quality of life.

The present methods provide a genetic test for late-onset AD. The current course of treatment focuses on slowing progression of the disease as there is no cure. This strategy involves changing lifestyles, diets, and may include medication. Identifying those at risk early should allow for aggressive treatment that hopefully extends the quality of life for late-onset AD patients.

The present methods may be used for diagnosing or prognosing late-onset Alzheimer's disease in a subject. Late-onset AD is typically recognized as AD which occurs in patient >65 years of age. Late-onset AD accounts for >95% of all AD cases.

The methods may involve: (a) directly or indirectly detecting the presence or absence of an apolipoprotein E type 4 (ApoE4) isoform or DNA encoding ApoE4 in a sample from a patient; (b) assessing the patient's gender; and (c) directly or indirectly detecting the presence or absence of a luteinizing hormone receptor (LHR) polymorphism referred to herein as “LHR2” (which corresponds to CC or GG for rs4073366), a follicle-stimulating hormone (FSH) polymorphism referred to herein as “FSH1” (corresponding to CC or CT for rs6169), or both. All three factors (a), (b), and (c) may be used to assess whether the subject is afflicted with Alzheimer's disease or whether the patient is at risk for developing Alzheimer's disease.

In some embodiments, individuals who are female and possess an ApoE4 allele (i.e., one or both E4 alleles) and the LHR2 polymorphism (CC or GG for rs4073366) may be at 100% risk for developing AD. This group may represent ˜23% of the AD population, or currently ˜783,000 of the 4.5 million AD individuals in the US (assuming a female:male ratio of 2:1). Individuals who are female and possess an ApoE4 allele and the FSH1 polymorphism (CC or CT for rs6169) may be at 90% risk for developing AD. This group may represent ˜30% of the AD population, or currently ˜810,000 of the 4.5 million AD individuals in the US. Individuals who possess an ApoE4 allele, the FSH1 polymorphism (CC or CT for rs6169) and the LHR2 polymorphism (CC or GG for rs4073366) may be at 87% risk for developing AD. This group may represent ˜23% of the AD population, or currently 1,018,000 of the 4.5 million AD individuals in the US. In other words, the disclosed methods may be utilized to identify ˜23% of females who have a 100% risk of AD (i.e., females who are ApoE4 positive and carry the LHR2 polymorphism); or ˜37% of females who have a >90% risk of AD (i.e., females who are ApoE4 positive and carry one or the other (or both) LHR2 and FSH1 polymorphisms); ˜23% of males who have a 87% risk for AD (i.e., males who are ApoE4 positive and carry both LHR2 and FSH1 polymorphisms).

In further embodiments, individuals who are male, homozygous (CC) at the location of the LHB2 polymorphism (rs4002462), and homozygous (CC) at the location of the FSHR2 polymorphism (rs974894), have a 100% risk of AD. Individuals who are female, homozygous (CC) at the location of the LHB2 polymorphism (rs4002462), and homozygous (TT) at the location of the FSHR2 polymorphism (rs974894), have an 83% risk of AD.

Conversely, methods of determining decreased risk for Alzheimer's disease in a subject also are disclosed. The methods may involve: (a) directly or indirectly detecting the presence or absence of an apolipoprotein E type 4 (ApoE4) isoform or DNA encoding ApoE4 in a sample from the patient; (b) assessing the patient's gender (e.g., where the complement gender is male); and (c) directly or indirectly detecting the presence or absence of a complement LH receptor polymorphism (i.e., genotype CG at LHR2), the complement of the FSH receptor polymorphism (i.e., genotype TT at FSH1), or both. For example, individuals who are female, homozygous (TT) at the location of the FSH1 polymorphism (rs6169), and homozygous (CG) at the location of the LHR2 polymorphism (rs4073366), have only a 10% risk of AD. Individuals who are female, possess at least one C-allelle at the location of the FSH1 polymorphism (rs6169), and are ApoE:e4⁻ have only a 16% risk of AD. Individuals who are male, homozygous (CC) at the location of the LHB2 polymorphism (rs4002462), and homozygous (TT) at the location of the FSHR2 polymorphism (rs974894), have only a 17% risk of AD.

Analysis of these gene-gene and gender interactions may be used to predict AD. These genetic association analyses support the role of steroidogenic modulators in the etiology of AD, especially since some of these SNPs increase the risk of AD, while others decrease the risk of AD. These analyses also demonstrate the utility of recursive partitioning as a way to identify multi-locus associations.

The foregoing methods may be performed together with other methods known in the art for assessing whether a patient has AD or whether a patient is likely to develop AD. For example, others have shown that estradiol levels in serum of women correlate with severity of AD, i.e. those with more post-menopausal serum estradiol are less likely to develop AD [see Manly J J et al., Neurology 2000 54:833-7]. Thus, in some embodiments, the foregoing methods may include measuring estradiol levels in a patient for assessing whether a patient has AD or whether a patient is likely to develop AD.

### Illustrative Embodiments

The following Embodiments are illustrative and are not intended to limit the claimed subject matter.

Embodiment 1. A method of assessing risk in a patient for developing Alzheimer's disease, the method comprising: (a) obtaining a nucleic acid sample from the patient; (b) detecting mutations in a plurality of genes of the nucleic acid sample, wherein the plurality of genes encode gene products that function in steroid biosynthesis.

Embodiment 2. The method of embodiment 1, wherein the method identifies the patient as having at least about 70% risk for developing Alzheimer's disease.

Embodiment 3. The method of embodiment 1, wherein the method identifies the patient as having at least about 90% risk for developing Alzheimer's disease.

Embodiment 4. The method of embodiment 1, wherein the method identifies the patient as having no more than 30% risk for developing Alzheimer's disease.

Embodiment 5. The method of embodiment 1, wherein the method identifies the patient as having no more than 10% risk for developing Alzheimer's disease.

Embodiment 6. The method of any of embodiments 1-5, further comprising identifying sex of the patient.

Embodiment 7. The method of any of embodiments 1-6, wherein the patient is female.

Embodiment 8. The method of any of embodiments 1-6, wherein the patient is male.

Embodiment 9. The method of any of embodiments 1-8, further comprising determining whether the patient is homozygous or heterozygous for the APOE2, APOE3, or APOE4 allele.

Embodiment 10. The method of any of embodiments 1-9, wherein the patient is homozygous for the APOE2, APOE3, or APOE4 allele.

Embodiment 11. The method of any of embodiments 1-9, wherein the patient is heterozygous for the APOE2, APOE3, or APOE4 allele.

Embodiment 12. The method of any of embodiments 1-11, wherein step (b) comprises sequencing the sample.

Embodiment 13. The method of any of embodiments 1-11, wherein step (b) comprises hybridizing the sample with oligonucleotide probes for detecting the mutations.

Embodiment 14. The method of any of embodiments 1-13, wherein the method assesses whether the patient is at risk for late-onset Alzheimer's disease.

Embodiment 15. The method of any of embodiments 1-14, wherein step (b) comprises identifying a nucleotide in the sample at a nucleotide position associated with a single nucleotide polymorphism selected from rs4073366 and rs6169.

Embodiment 16. The method of embodiment 15, comprising identifying a nucleotide in the sample at a nucleotide position associated with rs4073366 and identifying a nucleotide in the sample at a nucleotide position associated with rs6169.

Embodiment 17. The method of embodiment 15 or 16, further comprising determining whether the patient is homozygous or heterozygous at the nucleotide position associated with rs4073366.

Embodiment 18. The method of any of embodiments 15-17, further comprising determining whether the patient is homozygous or heterozygous at the nucleotide position associated with rs6169.

Embodiment 19. The method of any of embodiments 15-18, comprising identifying a C at a position associated with single nucleotide polymorphism rs4073366, thereby indicating that the patient has a C-allele.

Embodiment 20. The method of any of embodiments 15-19, wherein the patient is homozygous for the C-allele.

Embodiment 21. The method of any of embodiments 15-20, comprising identifying a G at a position associated with single nucleotide polymorphism rs4073366, thereby indicating that the patient has a G-allele.

Embodiment 22. The method of embodiment 21, wherein the patient is homozygous for the G-allele.

Embodiment 23. The method of any of embodiments 15-22, comprising identifying a C at a position associated with single nucleotide polymorphism rs4073366, thereby indicating that the patient has a C-allele; and identifying a G at a position associated with single nucleotide polymorphism rs4073366, thereby indicating that the patient has a G-allele and that the patient is heterozygous.

Embodiment 24. The method of any of embodiments 15-23, comprising identifying a C at a position associated with single nucleotide polymorphism rs6169, thereby indicating that the patient has a C-allele.

Embodiment 25. The method of embodiment 24, wherein the patient is homozygous for the C-allele.

Embodiment 26. The method of any of embodiments 15-25, comprising identifying a T at a position associated with single nucleotide polymorphism rs6169, thereby indicating that the patient has a T-allele.

Embodiment 27. The method of embodiment 26, wherein the patient is homozygous for the T-allele.

Embodiment 28. The method of any of embodiments 15-27, comprising identifying a C at a position associated with single nucleotide polymorphism rs6169, thereby indicating that the patient has a C-allele; and identifying a T at a position associated with single nucleotide polymorphism rs6169, thereby indicating that the patient has a T-allele and that the patient is heterozygous.

Embodiment 29. The method of embodiment 1, wherein step (b) comprises identifying a nucleotide in the sample at a nucleotide position associated with a single nucleotide polymorphism selected from rs4002462 and rs974894.

Embodiment 30. The method of embodiment 29, comprising identifying a nucleotide in the sample at a nucleotide position associated with rs4002462 and identifying a nucleotide in the sample at a nucleotide position associated with rs974894.

Embodiment 31. The method of embodiment 29 or 30, further comprising determining whether the patient is homozygous or heterozygous at the nucleotide position associated with rs4002462.

Embodiment 32. The method of any of embodiments 29-31, further comprising determining whether the patient is homozygous or heterozygous at the nucleotide position associated with rs974894.

Embodiment 33. The method of any of embodiments 29-32, comprising identifying a C at a position associated with single nucleotide polymorphism rs4002462, thereby indicating that the patient has a C-allele.

Embodiment 34. The method of embodiment 33, wherein the patient is homozygous for the C-allele.

Embodiment 35. The method of any of embodiments 29-34, comprising identifying a C at a position associated with single nucleotide polymorphism rs974894, thereby indicating that the patient has a C-allele.

Embodiment 36. The method of embodiment 35, wherein the patient is homozygous for the C-allele.

Embodiment 37. The method of any of embodiments 29-36, comprising identifying a T at a position associated with single nucleotide polymorphism rs974894, thereby indicating that the patient has a T-allele.

Embodiment 38. The method of embodiment 37, wherein the patient is homozygous for the T-allele.

Embodiment 39. The method of embodiment 1, wherein step (b) comprises identifying a nucleotide in the sample at a nucleotide position associated with a single nucleotide polymorphism selected from rs6166 and rs6521.

Embodiment 40. The method of embodiment 39, comprising identifying a nucleotide in the sample at a nucleotide position associated with rs6166 and identifying a nucleotide in the sample at a nucleotide position associated with rs6521.

Embodiment 43. The method of embodiment 39 or 40, comprising identifying an A at a position associated with single nucleotide polymorphism rs6166, thereby indicating that the patient has an A-allele.

Embodiment 44. The method of any of embodiments 39-43, further comprising determining whether the patient is homozygous or heterozygous at the nucleotide position associated with rs6521.

Embodiment 45. The method of embodiment 1, wherein step (b) comprises identifying a nucleotide in the sample at a nucleotide position associated with a single nucleotide polymorphism selected from rs974894 and Gpro.

Embodiment 46. The method of embodiment 45, comprising identifying a nucleotide in the sample at a nucleotide position associated with rs974894 and identifying a nucleotide in the sample at a nucleotide position associated with Gpro.

Embodiment 47. The method of embodiment 45 or 46, further comprising determining whether the patient is homozygous or heterozygous at the nucleotide position associated with rs974894.

Embodiment 48. The method of any of embodiments 45-47, further comprising determining whether the patient is homozygous or heterozygous at the nucleotide position associated with Gpro.

Embodiment 49. The method of any of embodiments 45-48, further comprising determining whether the patient is homozygous or heterozygous at the nucleotide position associated with rs974894 and determining whether the patient is homozygous or heterozygous at the nucleotide position associated with Gpro.

Embodiment 50. A kit comprising: (a) at least a first reagent for detecting a nucleotide in a sample at a nucleotide position associated with a single nucleotide polymorphism of rs4073366; and (b) at least a second reagent for detecting a nucleotide in a sample at a nucleotide position associated with a single nucleotide polymorphism of rs6169.

Embodiment 51. The kit of embodiment 50, further comprising: (c) at least a third reagent for detecting an APOE allele.

Embodiment 52. The kit of embodiment 50 or 51, wherein the first reagent detects whether the sample is homozygous or heterozygous at the nucleotide position associated with rs4073366.

Embodiment 53. The kit of any of embodiments 50-52, wherein the second reagent detects whether the sample has at least one C-allele of rs6169.

Embodiment 54. The kit of any of embodiments 50-53, wherein the first reagent detects whether the sample is homozygous or heterozygous at the nucleotide position associated with rs4073366; and the second reagent detects whether the sample has at least one C-allele of rs6169.

Embodiment 55. The kit of any of embodiments 50-54, wherein the first reagent comprises an oligonucleotide probe that specifically hybridizes to a C-allele or G-allele of rs4073366 and the second reagent comprises an oligonucleotide probe that specifically hybridize to a C-allele or T-allele of rs6169.

Embodiment 56. The kit of any of embodiments 50-55, wherein the first reagent comprises an oligonucleotide primer that specifically hybridizes to a C-allele or G-allele of rs4073366 and the second reagent comprises an oligonucleotide primer that specifically hybridize to a C-allele or T-allele of rs6169.

Embodiment 57. A kit comprising: (a) at least a first reagent for detecting a nucleotide in a sample at a nucleotide position associated with a single nucleotide polymorphism of rs4002462; and (b) at least a second reagent for detecting a nucleotide in a sample at a nucleotide position associated with a single nucleotide polymorphism of rs974894.

Embodiment 58. The kit of embodiment 57, wherein the first reagent detects whether the sample is homozygous for the C-allele of rs4002462.

Embodiment 59. The kit of embodiment 57 or 58, wherein the second reagent detects whether the sample is homozygous for the C-allele of rs974894 or whether the sample is homozygous for the T-allele of rs974894.

Embodiment 60. A kit comprising: (a) at least a first reagent for detecting a nucleotide in a sample at a nucleotide position associated with a single nucleotide polymorphism of rs6166; and (b) at least a second reagent for detecting a nucleotide in a sample at a nucleotide position associated with a single nucleotide polymorphism of rs6521.

Embodiment 61. The kit of embodiment 60, wherein the first reagent detects whether the sample has at least one A-allele of rs6166.

Embodiment 62. The kit of embodiment 60 or 61, wherein the second reagent detects whether the sample is homozygous or heterozygous at the nucleotide position associated with rs6521.

Embodiment 63. A kit comprising: (a) at least a first reagent for detecting a nucleotide in a sample at a nucleotide position associated with a single nucleotide polymorphism of rs974894; and (b) at least a second reagent for detecting a nucleotide in a sample at a nucleotide position associated with a single nucleotide polymorphism of Gpro.

Embodiment 64. The kit of embodiment 63, wherein the first reagent detects whether the sample is homozygous or heterozygous at the nucleotide position associated with rs974894.

Embodiment 65. The kit of embodiment 63 or 64, wherein the second reagent detects whether the sample is homozygous or heterozygous at the nucleotide position associated with Gpro.

### Example

The following Examples are illustrative and are not intended to limit the claimed subject matter. Reference is made to Haasl et al., BMC Medical Genetics 2008, 9:37, the content of which is incorporated herein by reference.

Background and Introduction

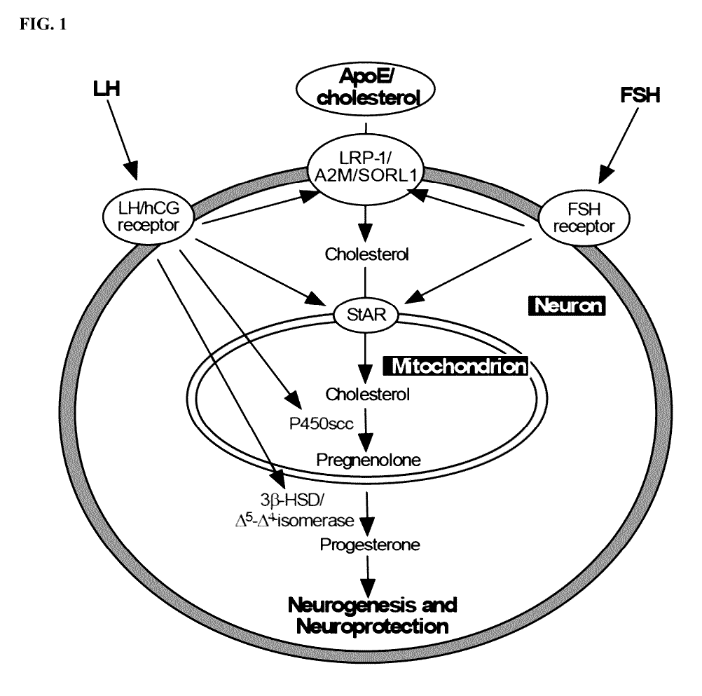

Genetic and biochemical studies have shown that the apolipoprotein E (APOE) ε4 allele is a major risk factor for late-onset Alzheimer's disease (AD), however approximately 50% of AD patients do not carry the allele. Since ApoE transports cholesterol for gonadotropin-regulated steroidogenesis, polymorphisms in a number of the components of the steroidogenic pathway (see FIG. 1) were studied, including LH beta-subunit (LHB), its receptor (LHCGR or LHR), GnRH ligand, its receptor (GnRHR), follicle-stimulating hormone (FSH), its receptor (FSHR), steroidogenic acute regulatory protein (STAR) and α2-macroglobulin (A2M), for their association with AD. As shown in FIG. 1, cholesterol used to produce neurosteroids is first bound extracellularly by ApoE which then enters the cell via endocytosis mediated by LRP-1, A2M, and SORL1. Transport into the mitochondria is then mediated by STAR and allows the subsequent conversion to pregnenolone, progesterone, and downstream neurosteroids by a variety of enzymes. Many of the intracellular processes are promoted by LH, FSH, or both.

Steroidogenic pathway members (ApoE, α-2-macroglobulin, androgen receptor, estrogen receptor) have been genetically and biochemically linked to AD. ApoE appears to be the greatest single risk factor. Genetic [see Haasl, R J et al., BMC Medical Genetics 2008, 9:37] and biochemical [see Bowen R L et al., J Neuroendocrinol 2000, 12(4):351-354; Bowen R L et al., J Biol Chem 2004, 279(19):20539-20545; and Casadesus G et al., Biochem Biophys Acta 2006] evidence also link LH and its receptor to AD. Here, experiments were performed to identify multi-locus associations to AD for components of the steroidogenic pathway.

Materials and Methods

Case-Control Setup.

The National Cell Repository for Alzheimer's Disease (NCRAD; University of Indiana, Bloomington, Ind.) provided total DNA samples from 100 control patients (negative for AD and other neurodegenerative diseases), and 100 late-onset AD patients (negative for other neurodegenerative diseases). All samples were obtained from North American Caucasian subjects. Control and AD groups comprised 50 female and 50 male patients each; all samples were derived from individuals ≧75 years of age, and all AD samples were acquired from individuals whose AoO was ≧75. Mean age of the control group was 84.73±4.61 years, while mean age of the AD group was 81.95±5.69 years. Among the AD samples, AoO was 79.18±3.47 years for males and 80.26±5.07 years for females. Direct sequencing of APOE, LHB (promoter, signal, and coding regions), LHCGR (exons 1, 10, and 11), GNRH (promoter, exon 1), GNRHR (exons 1, 2, and 3), FSH (exon 3 and 3′ untranslated region), FSHR (exon 10, introns 1 and 8), STAR (exon 7, intron 1), and A2M (exon 24) was performed using the primer pairs listed in Table 1. These amplified fragments contained at least one previously reported polymorphism with heteozygosity >5%. In addition, 40 samples kindly provided by the Sanders-Brown Center on Aging at the University of Kentucky, Lexington, Ky. were genotyped for APOE, LHB, and LHCGR. Cycle sequencing products were run on an ABI 3730 XL DNA analyzer at the University of Wisconsin Biotechnology Center (Madison, Wis.) and the resultant chromatograms were analyzed with FinchTV v1.4 (Geospiza, Seattle, Wash.). This study was carried out with IRB approval from the Health Sciences Institutional Review Board of the University of Wisconsin.

Data Analysis.

The dataset was analyzed using an array of analytic methods as previously described [see Ashley-Koch A E et al., Ann Hum Genet 2006, 70(Pt 3):281-292; and Haasl, R J et al., BMC Medical Genetics 2008, 9:37]. Interactive and main effects were tested, treating convergence of results from distinct analyses as the best evidence for association with AD. Allele and genotype counts were used in the following analyses: (1) χ₂ tests for main effects of individual polymorphisms; (2) tests of each locus for Hardy-Weinberg equilibrium; (3) tests of combinations of two loci for linkage disequilibrium (LD); (4) test for gene-gene interactions using multi-factor dimensionality reduction (MDR); (5) tests for interactions using logistic regression (LR), and; (6) tests for association with age of onset using one-way ANOVA. Finally, recursive partitioning (RP) was utilized to further understand and explore the interactions identified with these analyses. This allowed the identification of 3- and 4-factor interactions. To control for heterogeneity, the dataset was stratified according to gender and the same analyses was applied. For each bi-allelic locus, four genotype models were analyzed in tests for interactive effects: co-dominant, allele 1 dominant, allele 2 dominant, and over-dominant. By utilizing this schema: (1) the possibility of heterozygote advantage was addressed, and; (2) both alleles were tested for dominance, where no prior knowledge of which allele might carry the risk was known.

For each sample, genotype and demographic data were entered into a MySQL relational database, enabling the quick identification of samples meeting an array of criteria. APOE genotype, 9 previously reported polymorphisms of LHB, 5 of LHCGR, 3 of GnRH, 5 of GnRHR, 4 of FSH, 19 of FHSR, 2 of StAR, and 1 of A2M, were scored. For each polymorphism, allelic and genotypic frequencies of the AD and control groups were calculated. Additionally, both groups were stratified by gender and gender-specific allelic and genotypic frequencies were calculated. Four separate genotype models were used in tests for main and interactive effects of bi-allelic loci. For example, the following models would be used for a locus that varied between alleles B and b: (1) co-dominant (BB, Bb, bb); (2) B dominant (BB or Bb vs. bb); (3) b dominant (BB vs. Bb or bb), and; (4) over-dominant (BB or bb vs. Bb). For the tri-allelic APOE, an ‘ε4 dosage’ model (genotypes grouped by the number of ε4 alleles) and an ‘ε4 positive’ model (ε4 allele present or not) were used in analyses. A novel polymorphism was discovered in intron 8 of FSHR. This locus was thus found to be tri-allelic and for each of the 4 models above, the novel genotype (CC instead of AA, AG, or GG) was treated as a separate group.

To test for the association of individual polymorphisms with AD (main effects), χ² tests of allele and genotype counts were performed. Individual polymorphisms were tested for association with age of onset in the AD group using one-way ANOVA performed in Minitab. The program Genetic Data Analysis (GDA) was used to test each polymorphic locus for Hardy-Weinberg Equilibrium (HWE).

An analysis was performed that utilized a combination of linkage disequilibrium (LD), multi-factor dimensionality reduction (MDR), logistic regression (LR), and recursive partitioning (RP) analyses. The program Genetic Data Analysis (GDA) was used to test combinations of two loci for LD. In all tests for LD, genotypes were preserved in order to prevent significant deviations from HWE at a single locus from contributing to the measure of LD. Polymorphisms were excluded from multi-locus tests of association if they were found to be invariant in the dataset or in complete LD with an already included locus. MDR was performed using MDR Software²¹, which output the best 1-, 2-, 3-, and 4-factor models for a given dataset using 10-fold cross-validation (CV). Given the weight APOE carries as a single risk factor for AD, MDR was also run using APOE-free datasets in order to detect any interactions independent of APOE. An interaction model was considered significant if it was selected as the best model in 5 or more of the CV runs and exhibited a testing accuracy of >0.5 in 7 or more CV runs. Multi-locus combinations exhibiting LD (at a significance of p≦0.05) and significant multi-locus models discovered using MDR were input as disease models in LR analyses performed in Minitab.

The results of the LD and MDR analyses were used to select candidate pairs of loci as inputs to LR analysis. This form of model selection for LR was necessary because a lack of several multi-locus combinations made backward model selection impossible and the lack of significant main effects in most loci examined made forward model selection impractical. In addition to models identified as significant from the MDR analysis, any locus pair displaying linkage at p<0.05 in the AD group but not in the control group was chosen for LR analysis, as well as a few pairs with strong linkage in the AD group (p<0.005) but mild linkage in the control group (p>0.01).

The large number of tests performed overall meant that a correction to balance type I and type II error was a significant concern. A modified FDR approach was utilized [see Haasl, R J et al., BMC Medical Genetics 2008, 9:37, 63; and Benjamin Y et al., Behav Brain Res 125, 279-84 (2001)]. Because a multi-locus combination was only tested with LR if LD, MDR, or both were suggestive of its association with AD, only a subset of the possible two-locus LR tests were performed, and a different number of tests were performed on the male, female, and combined datasets. Because a multi-locus combination was only tested with LR if LD, MDR, or both analyses were suggestive of its association with AD, only a subset of the total array of possible LR tests were actually performed and the total, male, and female datasets were subjected to a different total number of tests: 614, 605, and 625 tests, resulting in modified FDR α levels of 0.0071, 0.0072, and 0.0071, respectively, as shown in Table 6.

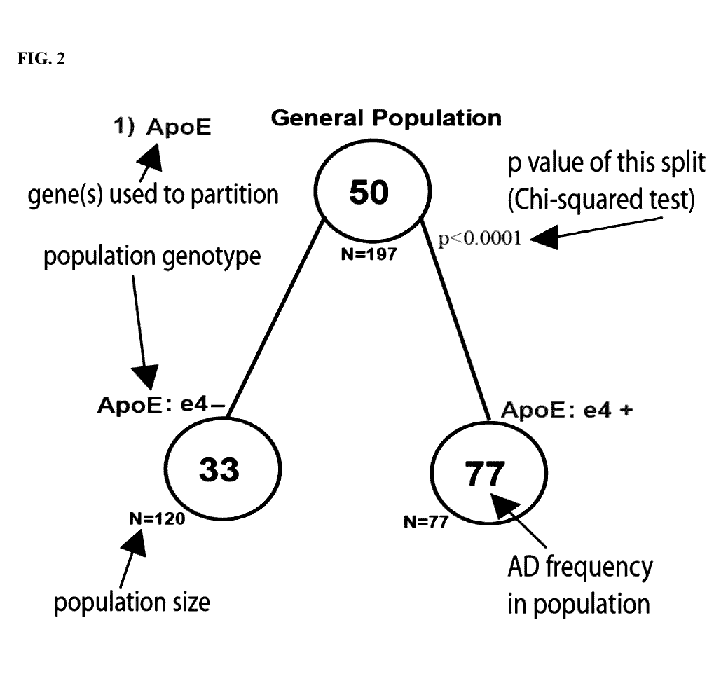

Finally, RP was used to examine the nature of the interactions identified in two or more of the three previous tests. As it was used here, RP was primarily a way of viewing the genetic dataset to view the effects of multiple factors (genotypes, gender). While statistical tests allow the precise calculation of the significance of an observed trend, the resultant numerical descriptors (p values, odds ratios, etc.) do not often give a full picture of what indeed the trend is. As an example of RP, FIG. 2 shows the information contained in a one-level recursive partitioning ‘tree.’ The tree is composed of nodes each representing one population with size (N) given below the node. The single parent node at the top of the tree, designated ‘General Population,’ has been split (partitioned) into two daughter nodes, representing two exhaustive and non-overlapping subsets of the parent population: note that their N values sum to that of the parent's. Only one such split has been performed in this tree, and the gene used to determine which daughter population a given member of the parent population would be assigned to is listed in the upper left corner, APOE.

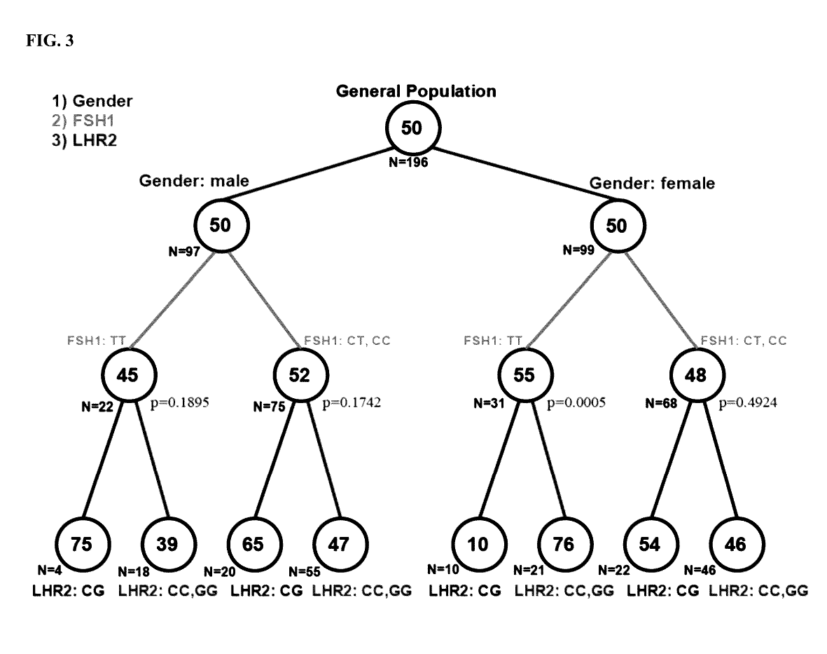

As shown in FIG. 2, each circle in the RP tree represents a study population. The number in the circle indicates the % incidence of Alzheimer's in the group, while the size of the population is indicated below the circle. If a population is split into daughter populations, the characteristic (genotype or gender) used to identify each sub-population is shown next to its circle. A relatively large difference between the AD incidence of the sub-populations indicates a role for the polymorphism/gene in AD. The significance of this role is estimated with a chi-squared test whose p-value is shown adjacent to the parent population. Subsequent splits can be performed on the daughter populations to identify multi-locus interactions, as in FIGS. 3-12. This example shows only one split, identifying two sub-populations of the general sample by their ApoE ε4status, either ε4+ or ε4−. The sharp difference in AD incidence between the two daughter populations (33 vs. 77) indicates a significant role for ApoE ε4 status, as indicated by the small p-value.

As described above, the population contained 200 samples, 50/50 AD and control. Those samples for which APOE genotype was missing were excluded from the tree, so the general population was 197 people, of whom 120 are ε4 negative and 77 are ε4 positive. The large number inside the circle represents the AD incidence of that population, an estimate of its risk. Thus APOE ε4 status was used to segregate a population with 50% AD incidence into daughter populations of 33% and 77% incidence for APOE ε4 negative and positive respectively. This large split in frequency is indicative of an important role for APOE ε4 in AD. While the size (N) of the daughter populations must sum to that of the parent population, there will always be one daughter with AD incidence lower than the parent incidence and one with higher (unless all daughters have the same incidence as the parent). This tree performs a binary split on the parent population, but more than two daughters can be created (i.e. genotype CC, CT, TT). Subsequent splits can also be performed on the set of daughter populations using a second gene, identifying granddaughter populations who are, for example, ε4 positive and carry one of three FSH1 genotypes. Recursive partitioning is not by itself a statistical technique, but the significance of the role for a particular gene implied by a given partition can be assessed with a χ² test, simply counting AD positive and AD negative populations for each daughter node. For this key, and among the general population, ε4 status is observed to be strongly predictive of AD risk, with p<0.0001.

Results

Analysis of Single-Locus, Main Effects.

Twenty-six loci were scored comprising 19 polymorphisms in FSHR, 4 in FSH, 2 in StAR, and 1 in A2M, and the resultant data were combined with the data we previously reported for the same population covering ApoE genotype, 9 polymorphisms in LHB, 5 in LHCGR, 3 in GnRH, and 5 in GnRHR [see Haasl, R J et al., BMC Medical Genetics 2008, 9:37, and unpublished data). Of these 50 loci, 18 were excluded from the data analyses due to invariance or covariance with another locus (Table 2 lists the analyzed loci, Table 3 lists the excluded loci and reason for exclusion). No significant differences were found in either the allele frequencies or the genotypes of the AD and control populations at any single locus scored in the current study, i.e. no single locus effects were found in FSHβ, FSHR, A2M, or StAR or in GnRH and GnRHR (unpublished results), or in LHβ from a previous analysis [see Haasl, R J et al., BMC Medical Genetics 2008, 9:37]. As previously reported, a single locus effect was found for LHCGR [see Haasl, R J et al., BMC Medical Genetics 2008, 9:37].

Analysis of Gene-Gene Interactions, LD Analyses.

Seven new pairs of loci were significantly associated with AD in both genders for the loci measured in this study; five pairs of loci were significantly associated in males and seven pairs in females. Of the locus pairs measured in this study, there were no overlapping locus pairs between the male and female groups, while FSHR6/FSH2 and FSHR8/FSH2 was significant in both males and the combined population, and LHB4/StAR and LHB5/StAR were significant in both females and the combined population. Table 4 lists the locus pairs chosen for LR testing on the basis of LD results in the current and previous study [see Haasl, R J et al., BMC Medical Genetics 2008, 9:37]. Locus pairs in bold meet the more stringent threshold for significance after correcting for multiple tests; these are also indicated in Table 5 which lists the significant multi-locus interactions detected using one or more of LD, MDR, and LR analyses.

Analysis of Gene-Gene Interactions, MDR Analyses.

In the current study ApoE was the best single factor model and was significantly associated with AD. In the total dataset (including ApoE and both genders) the only other significant model was ApoE, Gpro, and FSHR11. In the male dataset, ApoE and LHR2 was significant while for females both the 2 factor model ApoE and FSH2 as well as the 3 factor model ApoE, LHR2, and FSH2 were significant.

Also analyzed were the combined and gender-stratified datasets in the absence of ApoE data to look for effects that were masked by the strong ApoE ε4 effect. No significant models were detected in the combined dataset. In the male dataset, FSHR11 was significant as a single factor model, and for females a two factor model was significant: FSH1 and LHR2. Table 5 lists all significant models suggested by MDR for this and a previous study [see Haasl, R J et al., BMC Medical Genetics 2008, 9:37].

Analysis of Gene-Gene Interactions, LR Analyses.

Locus pairs indicated as significant from the MDR tests were the inputs to LR analysis. Linkage disequilibrium was also used to suggest candidate locus pairs for LR analysis, with pairs showing linkage at p<0.05 in the AD group but not in the control group chosen for LR testing. In addition, a few pairs with strong linkage in the AD group (p<0.005) and mild linkage in the in the control group (0.01<p<0.05) were tested with LR. All candidate locus pairs derived from LD results are listed in Table 5. LR testing at the modified FDR level confirmed seven multi-gene associations suggested by LD and MDR analysis: four in females, one in males, and two in the combined population. The most significant was in the female population where a significant interaction was found between LHR2 and FSH1 (the strongest result treated both genes with the recessive model, p<0.001 and OR=44.12 (5.26-370.22). LHR2 was also associated with FSH2 in females (the strongest result used the recessive model for LHR2 and the dominant model for FSH2, p=0.003 and OR=0.04 (0.00-0.32), while LHR4 interacted with both FSHR11 and FSHR13 in females (using the co-dominant model for LHR4 and the over-dominant model for both FSHR11 (p=0.007, OR=0.07 (0.01-0.49)) and FSHR13 (p=0.006, OR=0.07 (0.01-0.47)). In the male population a significant association was found between LHB2 and FSHR2 (the strongest result used the LHB2 over-dominant genotype model and the FSHR2 co-dominant genotype model, p=0.006 and OR=60.00 (3.17-1137.01).

In the general population two locus pairs demonstrated significant association: LHB4 and FSHR13 (the strongest result used the LHB4 over-dominant genotype model and the FSHR13 co-dominant genotype model, p=0.006 and OR=0.08 (0.01-0.48)), and GPRO and FSHR2 (the strongest result use the over-dominant model for both genes, giving p=0.003 and OR=0.16 (0.05-0.54)). Table 5 lists significant interactions identified with LR analysis in this study and as previously observed [see Haasl, R J et al., BMC Medical Genetics 2008, 9:37].

RP Analysis.

Recursive partitioning identified three factors as exacerbating the risk associated with APOE ε4 alleles. Two of these three are single locus genotypes (CT or CC at FSH1, CC or GG at LHR2), the third is gender (female). As shown in FIG. 3, FSH1 genotype does not modulate AD risk by itself in either gender. Females with FSH1: TT, however, show a strong protective effect (p=0.0005) of LHR2 heterozygosity (LHR2: CG), while males with FSH1: TT show a trend in the opposite direction that fails to reach significance, where LHR2 heterozygosity leads to a higher AD incidence than the complementary genotypes (LHR2: CC or GG).

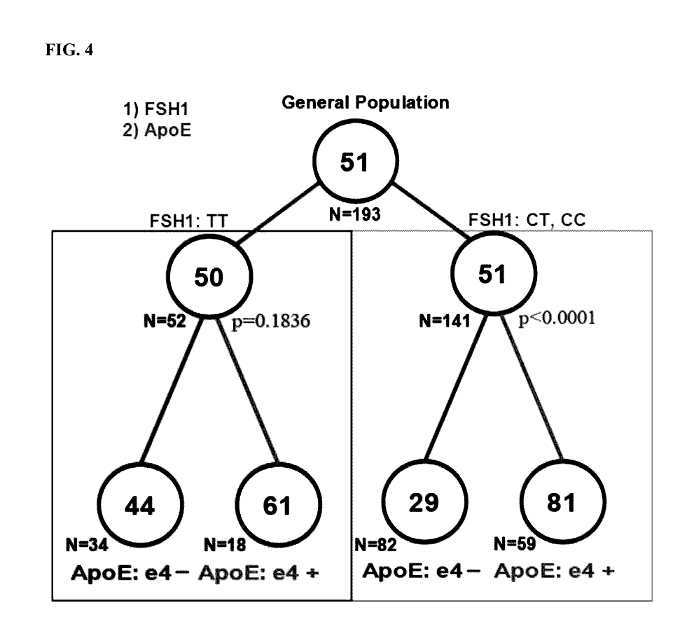

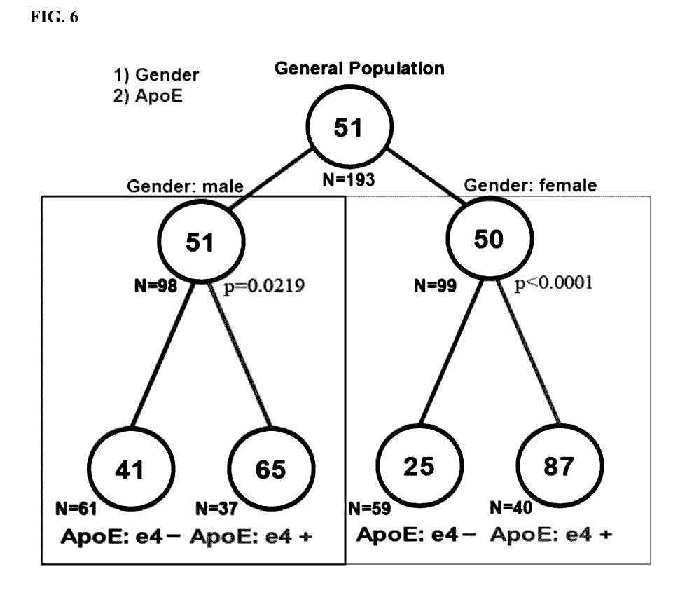

Members of the APOE ε4 positive population with one or more of these factors have an increased AD risk compared to those who are APOE ε4 positive but have none of them. As shown in FIG. 4, ApoE ε4 status strongly predicts AD risk among FSH1: CT, CC genotypes (p<0.0001) but fails to reach significance among FSH1: TT (p=0.1836). FSH1: TT therefore reduces ε4-associated risk. As shown in FIG. 5, ApoE ε4 status strongly predicts AD risk among LHR2: GG,CC genotypes (p<0.0001) but is less significant among LHR2: CG (p=0.1344). LHR2: CG therefore reduces ε4-associated risk. Note that the sample population genotyped for LHR2 is larger than for other genes. (See Materials and Methods section). As shown in FIG. 6, ApoE ε4 status strongly predicts AD risk among females (p<0.0001) but is only marginally significant among men (p=0.0219). Being male therefore reduces ε4-associated risk.

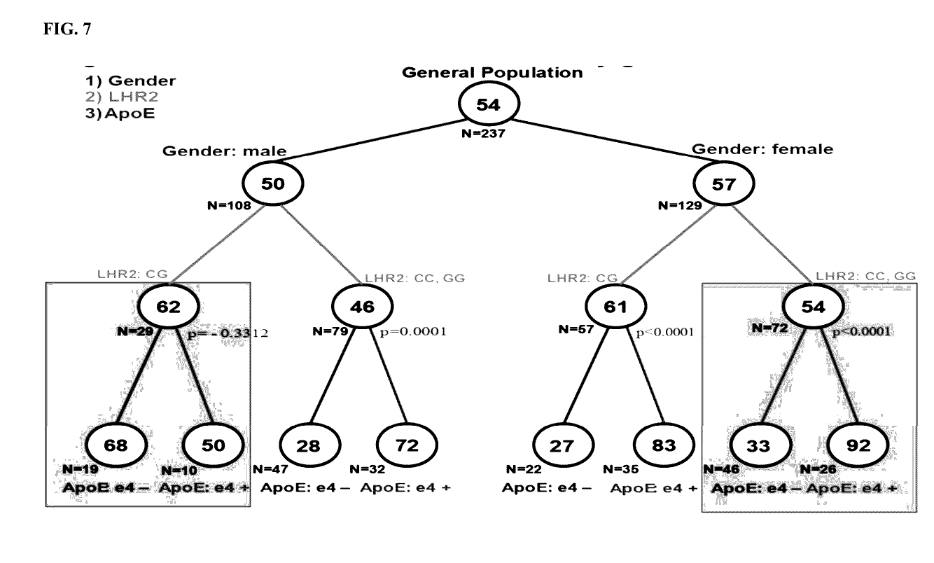

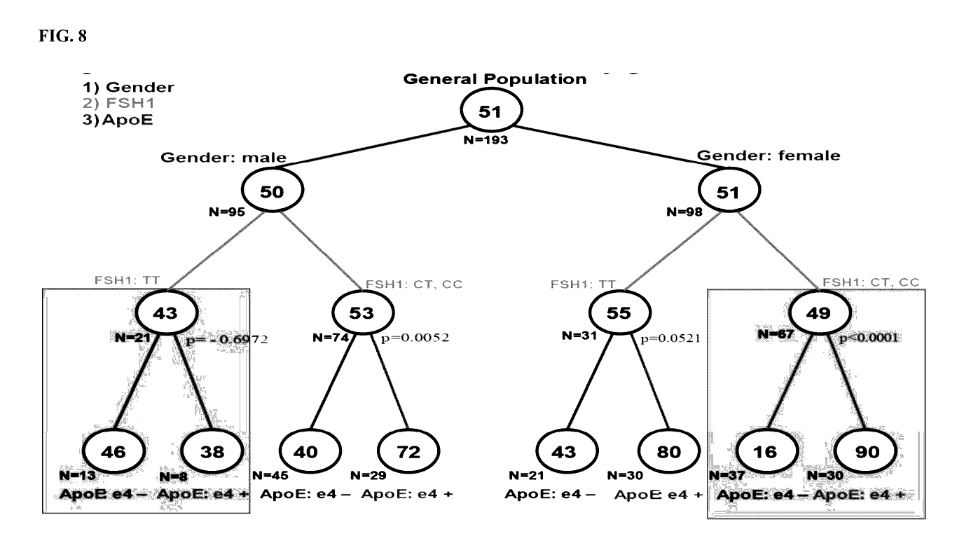

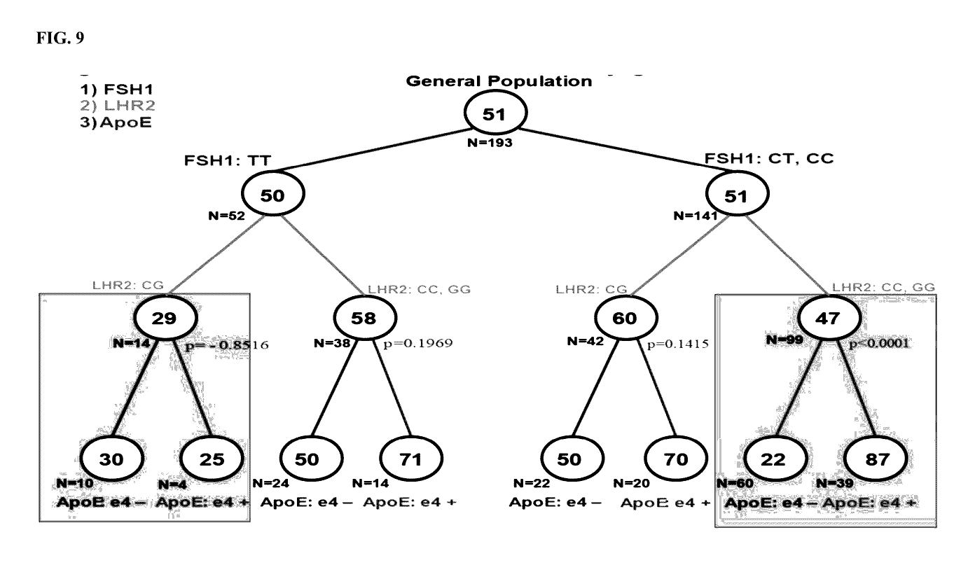

Furthermore, these factors are additive, such that having two or more of the three factors raises the risk among the APOE ε4 positive population to between 87-92%. As shown in FIG. 7, in the population with both of the factors that reduce ε4-associated risk (males; LHR2:CG), ε4-associated risk is reversed, with the ε4+ population having a lower AD incidence than the ε4− population, (50% vs. 68%) though the trend is not significant. In the population with both complementary factors (females; LHR2: CC, GG), the normal ε4 trend is observed to be exacerbated, i.e., the difference in AD risk between ε4+ and ε4-populations is greater than in the general population (92% vs. 33%, respectively). As shown in FIG. 8, in the population with both of the factors that reduce ε4-associated risk (males; FSH1:TT), ε4− associated risk is reversed, with the ε4+ population having a lower AD incidence than the ε4− population, (38% vs. 46%) though the trend is not significant. In the population with both complementary factors (females; FSH1: CT, CC), the normal ε4 trend is observed to be exacerbated, i.e., the difference in AD risk between ε4+ and ε4− populations is greater than in the general population (90% vs. 16%, respectively). As shown in FIG. 9, in the population with both of the factors that reduce ε4− associated risk (FSH1: TT; LHR2:CG), ε4-associated risk is reversed, with the ε4+ population having a lower AD incidence than the ε4− population, (25% vs. 30%) though the trend is not significant. In the population with both complementary factors (FSH1: CT, CC; LHR2: CC, GG), the normal ε4 trend is observed to be exacerbated, i.e., the difference in AD risk between ε4+ and ε4− populations is greater than in the general population (87% vs. 22%, respectively).

The existence of these three factors increasing the AD risk associated with the APOE ε4 positive population necessarily implies the existence of a set of three complementary factors which decrease the APOE ε4 associated risk: 1) TT at FSH1, 2) CG at LHR2, 3) male. Members of the APOE ε4 positive population with one or more of these complementary factors have a decreased AD risk compared to those who are APOE ε4 positive but have none of them. (See FIGS. 4-6). Having two or more of the three complementary factors is sufficient to reverse the association, lowering the risk among the APOE ε4 positive population to about 40%, below that of the APOE ε4 negative population having two or more of the three complementary factors. (See FIGS. 7-9).

These two loci also exhibit an interaction that modulates AD risk directly (independent of APOE ε4 status) in a gender-dependent manner—this is the interaction detected by LR analysis. FSH1: TT genotype trends toward a protective effect in males with LHR2:CG and in females with LHR2:CC or GG, while FSH1:TT was deleterious in males with LHR2:CC or GG and in females with LHR2:CG. (See FIG. 3).

The LR analysis suggested another gender-dependent two locus interaction: LHB2/FSHR2 in males. RP analysis confirmed and described this interaction: in males with LHB: CC, FSHR2 ‘G’ alleles are deleterious, while in males with LHB2: TT, FHSR2 genotype has no effect. Examining this interaction highlighted one of the strengths of RP analysis, which, in addition, revealed an interesting trend in females which is similar (FSHR2 genotype is neutral in the LHB2: TT population but predictive for the LHB2: CC group) but reversed in direction (risk increasing haplotypes in men are risk decreasing in women and vice versa). LR analysis did not identify this aspect of the interaction between FSHR2 and LHB2 because the actual predictive power is limited to smaller groups (women with LHB2: CC and men with LHB2: CC, but not in the combined population) the statistical significance is limited (p=0.0892) even while the overall interaction is apparent from the RP analysis.

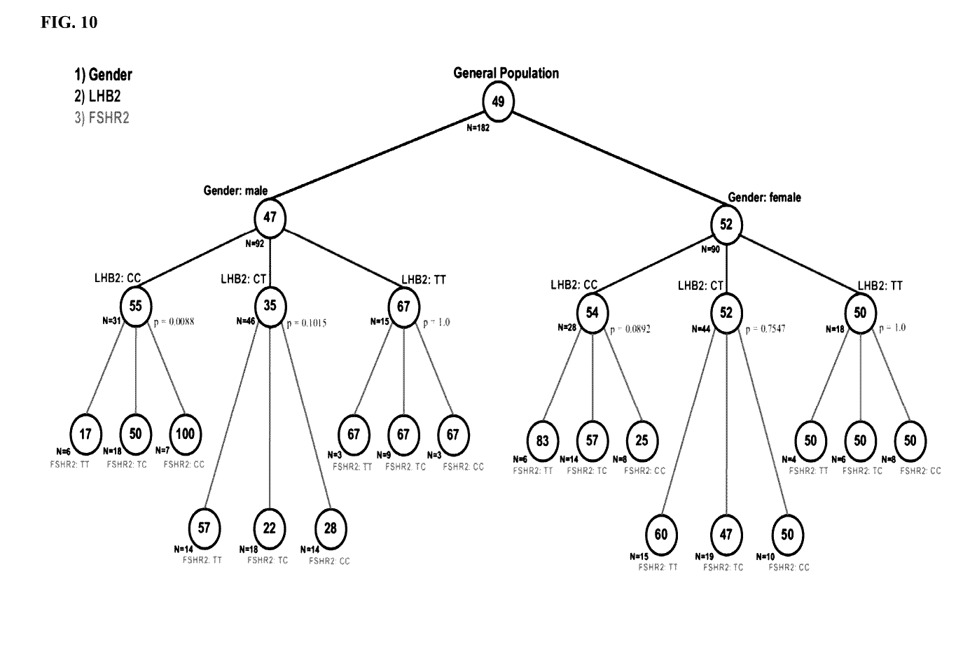

As shown in FIG. 10, in males, LHβ2 genotype does not by itself significantly affect AD risk: every daughter node of the general population has an AD incidence close to 50%. However, among the male LHβ2: CC population, FSHR2 ‘C’ alleles are strongly predictive of AD (p=0.0088) while they have no predictive power among the male LHβ2: TT population (p=1.0), nor significant predictive power among the general population. In females, too, LHβ2 does not affect AD risk by itself. However, among the female LHβ2: CC population, FSHR2 ‘T’ alleles trend toward predicting AD (p=0.0892), while FSHR2 genotype is again completely neutral among the female LHβ2: TT population (p=1.0).

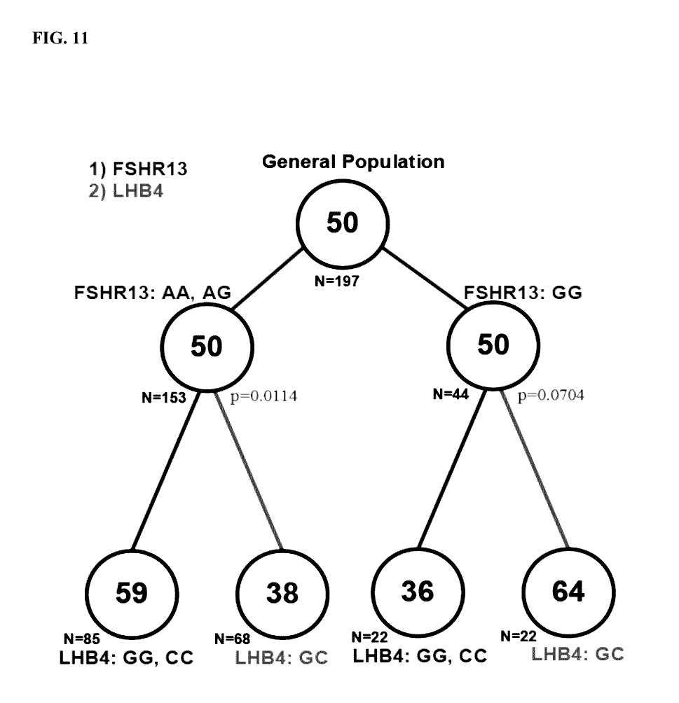

Both pairs of genes identified in LR analysis as significantly associated in the general population showed clear patterns on their own which did not interact with either sex or ApoE status. Heterozygosity at LHβ4 is protective among carriers of ‘AA’ and ‘AG’ alleles at FSHR13 but deleterious otherwise. As shown in FIG. 11, FSHR13 is not predictive by itself, but amongst the FSHR13: AA, AG population, LHβ4 homozygosity (CC or GG) confers significant AD risk compared to LHβ4 heterozygosity (p=0.0114), while in the FSHR13: GG population LHβ4 homozygosity is mildly protective (p=0.0704) compared to the heterozygote.

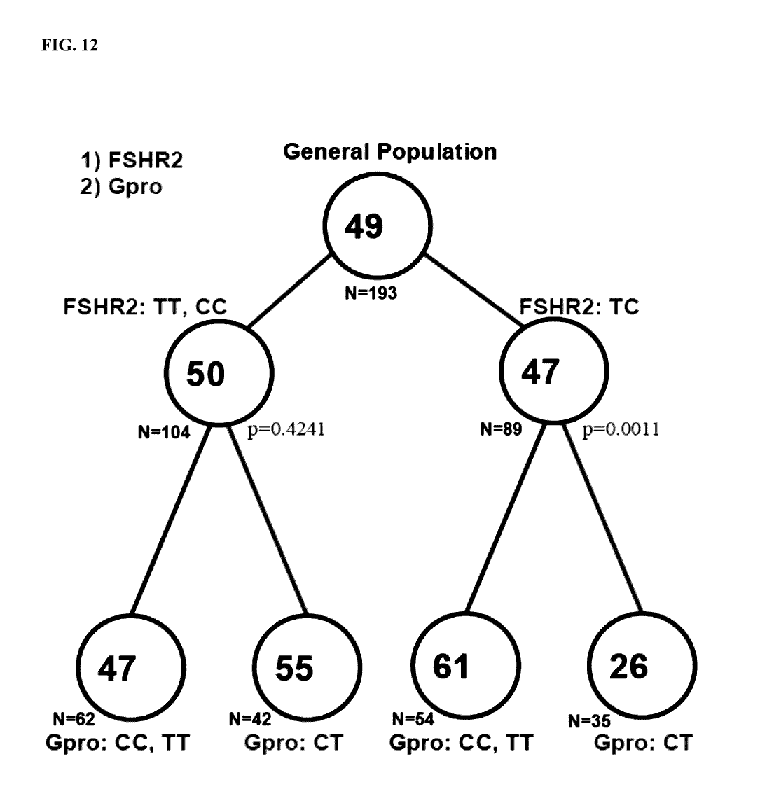

Heterozygosity at Gpro is protective among FSHR2 heterozygotes and insignificantly deleterious otherwise. As shown in FIG. 12, neither GPRO nor FSHR2 are significantly predictive on their own, but among those with FSHR2: CT, heterozygosity at GPRO is significantly protective (p=0.0011) compared to either homozygote (GPRO: CC or TT).

In the foregoing description, certain terms have been used for brevity, clearness, and understanding. No unnecessary limitations are to be implied therefrom beyond the requirement of the prior art because such terms are used for descriptive purposes and are intended to be broadly construed. The different compositions and method steps described herein may be used alone or in combination with other compositions and method steps. It is to be expected that various equivalents, alternatives and modifications are possible.

TABLE 1
Primer pairs used to amplify portions of APOE, LHβ, LHCGR, GNRH, GNRHR, FSHβ, FSHR, STAR and A2M.

| Region | Forward Primer | Reverse Primer | SEQ ID NOS. |
|---|---|---|---|
| APOE exon 4 | 5′-GGCACGGCTGTCCAAGGA-3 | 5′-CTGGCGGATGGCGCTGAG-3′ | SEQ ID NO: 8, 9 |
| LHβ 5′ | 5′-GTTACCCCAGGCATCCTATC-3′ | 5′-CCATTCCCCAACCGCAGG-3′ | SEQ ID NO: 10, 11 |
| LHβ 3′ | 5′-GGTCCTGAATAGGAGATGCCA- | 5′-CGGGGTGTCAGGGCTCCA-3′ | SEQ ID NO: 12, 13 |
| LHCGR axon 1 | 5′-CACTCAGAGGCCGTCCAAG-3′ | 5′-GGAGGGAAGGTGGCATAGAG-3′ | SEQ ID NO: 14, 15 |
| LHCGR exon 10 | 5′-ACAGTCAGGTTTAGCCTGAA-3′ | 5′-CTTCTGAGTTTCCTTGCATG-3′ | SEQ ID NO: 16, 17 |
| LHCGR exon 11 | 5′-CAGAAAATCCCTTACCTCAAGC-3′ | 5′-GGTTTAAGAACAATTCAATAATGCAG-3′ | SEQ ID NO: 18, 19 |
| GnRH promoter | 5′-ATAGAGGCAGCATTAGGCCTTACC-3′ | 5′-TGGATTCCCTTGAGGAAACCAGCA-3′ | SEQ ID NO: 20, 21 |
| GnRH 5′ | 5′-GAAGAATCCAAGAGCCAG-3′ | 5′-GCATTACTGCTGGCTGAACCATCT-3′ | SEQ ID NO: 22, 23 |
| untranslated | | | |
| region | | | |
| GnRH exon 1 | 5′-TCTGACTTCCATCTTCTGCAGGGT-3′ | 5′-AGTGCCTTATCTCACCTGGAGCAT-3′ | SEQ ID NO: 24, 25 |
| GnRH exon 2 | 5′-GCATTTGACAGCCCAAGGGCTAAA-3′ | 5′-AAGTGCCTTATCTACCTGGAGCA-3′ | SEQ ID NO: 26, 27 |
| GnRHR exon 1 | 5′-ACACAAGGCTTGAAGCTCTGTCC-3′ | 5′-AAGAGCAGCTTCATTCTTGAGAG-3′ | SEQ ID NO: 28, 29 |
| GnRHR exon 1B | 5′-ACACAGAAGAAAGAGAAAGGG-3′ | 5′-GCTGTTGCTTTTCAAAGCTAGG-3′ | SEQ ID NO: 30, 31 |
| GnRHR exon 1C | 5′-CTTTTCTCCATGTATGCCCCAG-3′ | 5′-AGACCTTATATCAAATTTAGATAGGA-3′ | SEQ ID NO: 32, 33 |
| GnRHR exon 2A | 5′-CTAGCAGAGTACCAAAGAGAAAACTT-3′ | 5′-AGGGATGATGAAGAGGCAGCTG-3′ | SEQ ID NO: 34, 35 |
| GnRHR axon 28 | 5′-TAGCAGACAGCTCTGGACAGAC-3′ | 5′-AAACTGCCCACAAATGACACT-3′ | SEQ ID NO: 36, 37 |
| GnRHR exon 3A | 5′-CACCTCTCTTTTCTCTATCCATCA-3′ | 5′-CCATAGATAAGTGCATCAAAGC-3′ | SEQ ID NO: 38, 39 |
| GnRHR exon 3B | 5′-CCTAGGAATTTGGTATTGGTTTG-3′ | 5′-ACATTTGTGTTAATCATTCCCAGA-3′ | SEQ ID NO: 40, 41 |
| FSHβ exon 3 | 5′-TGTTAGAGCAAGCAGTATTCAATTTCT-3′ | 5′-GTATGTGGCCTGAAATGTCCACTGAT-3′ | SEQ ID NO: 42, 43 |
| FSHβ 3′ | 5′-AGAGCAAGGTCAGCATCTTCAGCA-3′ | 5′-TTGCAGGAGCCTAGTAGCATGTGA-3′ | SEQ ID NO: 44, 45 |
| untranslated | | | |
| region | | | |
| FSHR intron 1 | 5′-TACAGAAATGCTGGTGTGGCTCCT-3′ | 5′-CCAAACAAAGCACCTGTTGTCCTC-3′ | SEQ ID NO: 46, 47 |
| FSHR intron 8 | 5′-TCCCTGTCATCCAGGAACCACTTT-3′ | 5′-TCTCAGCGGTGCCTTTCATGTAGT-3′ | 8E0 ID NO: 48, 49 |
| FSHR axon 10, | 5′-CCCACATTCAGGTTGTGGCAAGAT-3′ | 5′-GCTGCTGATGCCAAAGATGGGAAA-3′ | SEQ ID NO: 50, 51 |
| 5′ region | | | |
| FSHR axon 10, | 5′-TGTCAGTCTACACTCTGACAGC-3′ | 5′-GTGACATACCCTTCAAAGGCAAGA-3′ | SEQ ID NO: 52, 53 |
| 3′ region | | | |
| STAR intron 1 | 5′-ATGGAAGGCAGATTTCTGGACCCT-3′ | 5′-AAGCCTCAGCACTTACCGAGTAGA-3′ | SEQ ID NO: 54, 55 |
| STAR exon 7 | 5′-AGCTGATTAATGGGCCCTGGAAGA-3′ | 5′-CCCAATGTGTGTGTGTGTGTGTGT-3′ | SEQ ID NO: 56, 57 |
| A2M exon 24 | 5′-TGGCTTTGGAGAGCAGAATATGGT-3′ | 5′-GGAGGTTGGAGAGTGGATAGTTTCCT-3′ | SEQ ID NO: 58, 59 |

TABLE 2
Analyzed Steroidogenic Pathway Genes/Loci

| | | dbSNP | | |
|---|---|---|---|---|
| Gene | Designation | reference ID | Location | Type |
| FSH | FSH1 | rs6169 | Exon 3 | Synonymous SNP |
| | FSH2 | rs676349 | 3′ untranslated | Untranslated SNP |
| | | | region | |
| FSHR | FSHR1 | rs7590213 | Intron 1 | Intronic SNP |
| | FSHR2 | rs974894 | Intron 1 | Intronic SNP |
| | FSHR3 | rs974895 | Intron 1 | Intronic SNP |
| | FSHR4 | rs974896 | Intron 1 | Intronic SNP |
| | FSHR5 | rs11693287 | Intron 8 | Intronic SNP |
| | FSHR6 | rs759493 | Intron 8 | Intronic SNP |
| | FSHR7 | rs2284673 | Intron 8 | Intronic SNP |
| | FSHR8 | rs2268363 | Intron 8 | Intronic SNP |
| | FSHR9 | rs6545091 | Intron 8 | Intronic SNP |
| | FSHR10 | rs2268362 | Intron 8 | Intronic SNP |
| | FSHR11 | rs6165 | Exon 10 | Missense SNP |
| | FSHR12 | rs6167 | Exon 10 | Missense SNP |
| | FSHR13 | rs6166 | Exon 10 | Missense SNP |
| A2M | A2M | rs669 | Exon 24 | Missense SNP |
| STAR | STAR | rs3990403 | Exon 7 | Untranslated SNP |
| GNRH | GPRO | novel | promoter | promoter |
| | GX1 | rs6185 | Exon 1 | Missense SNP |
| ApoE | APOE | rs429358 | Exon 4 | Missense SNP |
| | | rs7412 | Exon 4 | Missense SNP |
| LHβ | LHB1 | rs3956233 | Intron 1 | Intronic SNP |
| | LHB2 | rs4002462 | Intron 1 | Intronic SNP |
| | LHB3 | rs1800447 | Exon 2 | Non-synonymous |
| | | | (vLH SNP 1) | SNP |
| | LHB4 | rs6521 | Exon 2 | Synonymous SNP |
| | LHB5 | rs1056914 | Exon 2 | Synonymous SNP |
| | LHB6 | rs2387588 | Intron 2 | Intronic SNP |
| LHR | LHR1 | rs4539842 | Exon 1 | 6 base |
| | | | | insertion/deletion |
| | LHR2 | rs4073366 | Intron 1 | Intronic SNP |
| | LHR3 | rs12470652 | Exon 10 | Non-synonymous |
| | | | | SNP |
| | LHR4 | rs2293275 | Exon 10 | Non-synonymous |
| | | | | SNP |
| | LHR5 | rs13006488 | Exon 11 | Synonymous SNP |

TABLE 3
Loci Excluded from Analysis

| | Reason for | dbSNP | | |
|---|---|---|---|---|
| Gene | exclusion | reference ID | Location | Type |
| FSH | covariance | rs506306 | 3′ | Untranslated SNP |
| | with FSH2 | | untranslated | |
| | | | region | |
| | covariance | rs506197 | 3′ | Untranslated SNP |
| | with FSH2 | | untranslated | |
| | | | region | |
| FSHR | covariant | rs7563620 | Intron 1 | Intronic SNP |
| | with FSHR1 | | | |
| | covariant | rs974897 | Intron 1 | Intronic SNP |
| | with FSHR4 | | | |
| | invariant | rs2898871 | Exon 10 | Missense SNP |
| | invariant | rs6168 | Exon 10 | Synonymous SNP |
| | invariant | rs28928870 | Exon 10 | Missense SNP |
| | invariant | rs12620825 | Exon 10 | Missense SNP |
| STAR | invariant | rs2070347 | Intron 1 | Intronic SNP |
| LHβ | invariant | rs5030775 | Exon 2 | Non-synonymous |
| | | | | SNP |
| | covariance | rs1800447 | Exon 2 | Non-synonymous |
| | with LHB3 | | | SNP |
| GNRH | invariant | rs35542850 | Exon 1 | Missense SNP |
| | invariant | rs6186 | Exon 2 | Missense SNP |
| GNRHR | invariant | rs35400155 | Exon 1 | Synonymous SNP |
| | invariant | rs4986942 | Exon 1 | Synonymous SNP |
| | invariant | rs13130501 | Exon 2 | Synonymous SNP |
| | invariant | rs13149772 | Exon 2 | Missense SNP |
| | invariant | rs28933074 | Exon 3 | Missense SNP |

TABLE 4
Loci exhibiting pairwise linkage disequilibrium at p ≦ 0.05 with bold-faced loci indicate a combination detected
at the α = 0.0071 level in an AD stratum but not in the corresponding control stratum.

| | | p-value of | | | p-value of | | | p-value of |
|---|---|---|---|---|---|---|---|---|
| Total | Loci | AD linkage | Female | Loci | AD linkage | Male | Loci | AD linkage |
| | GPRO, FSHR2 | 0.048 | | GX1, FSHR6 | 0.018 | | GPRO, FSHR2 | 0.008 |
| | GPRO FSHR7 | 0.031 | | GX1, FSHR8 | 0.020 | | GPRO, FSHR4 | 0.028 |
| | GPRO, FSHR10 | 0.038 | | APOE, FSH1 | 0.050 | | GPRO, FSHR7 | 0.015 |
| | GPRO, STAR | 0.005 | | APOE, STAR | 0.002 | | GPRO, FSHR10 | 0.049 |
| | GX1, LHR2 | 0.027 | | LHR1, STAR | 0.015 | | GPRO, FSHR12 | 0.013 |
| | GX1, STAR | 0.010 | | LHR2, FSHR2 | 0.017 | | LHR1, LHB1 | 0.029 |
| | LHR1, LHB1 | 0.017 | | LHR2, FSHR4 | 0.018 | | LHR1, LHB5 | 0.020 |
| | LHR3, STAR | 0.003 | | LHR2, FSH1 | 0.022 | | LHR2, APOE | 0.002 |
| | LHR4, FSH2 | 0.047 | | LHR2, FSH2 | 0.008 | | LHR5, LHB2 | 0.023 |
| | LHR4, STAR | 0.041 | | LHR2, STAR | 0.006 | | LHR5, LHB4 | 0.033 |
| | LHB2, STAR | 0.008 | | LHR2, FSHR12 | 0.028 | | LHR5, LHB5 | 0.028 |
| | LHB3, APOE | 0.000 | | LHR3, FSH2 | 0.033 | | LHB2, FSHR2 | 0.028 |
| | LHB4, FSHR13 | 0.032 | | LHR3, STAR | 0.013 | | LHB2, FSHR10 | 0.041 |
| | LHB4, STAR | 0.001 | | LHR4, STAR | 0.049 | | LHB2, STAR | 0.049 |
| | LHB5, STAR | 0.000 | | LHR4, FSHR11 | 0.007 | | LHB4, FSHR13 | 0.043 |
| | FSHR11, STAR | 0.036 | | LHR4, FSHR13 | 0.011 | | FSHR6, FSH1 | 0.002 |
| | FSHR13, STAR | 0.044 | | LHB1, FSHR10 | 0.032 | | FSHR6, FSH2 | 0.000 |
| | FSHR2, STAR | 0.029 | | LHB1, FSH1 | 0.046 | | FSHR7, FSH2 | 0.066 |
| | FSHR4, STAR | 0.027 | | LHB1, FSH2 | 0.033 | | FSHR8, FSH1 | 0.003 |
| | FSHR6, FSH1 | 0.012 | | LHB1, STAR | 0.006 | | FSHR8, FSH2 | 0.001 |
| | FSHR6, FSH2 | 0.002 | | LHB3, STAR | 0.043 | | | |
| | FSHR7, STAR | 0.021 | | LHB4, STAR | 0.004 | | | |
| | FSHR8, FSH1 | 0.028 | | LHB5, STAR | 0.003 | | | |
| | FSHR8, FSH2 | 0.004 | | FSHR2, A2M | 0.038 | | | |
| | FSHR10, STAR | 0.023 | | FSHR2, STAR | 0.011 | | | |
| | | | | FSHR4, STAR | 0.004 | | | |
| | | | | FSHR6, STAR | 0.034 | | | |
| | | | | FSHR7, STAR | 0.013 | | | |
| | | | | FSHR8, STAR | 0.036 | | | |
| | | | | FSHR9, STAR | 0.044 | | | |
| | | | | FSHR10, STAR | 0.017 | | | |
| | | | | FSHR11, STAR | 0.028 | | | |
| | | | | FSHR12, STAR | 0.036 | | | |
| | | | | FSHR13, STAR | 0.033 | | | |

TABLE 5
Significant Results from Multi-Locus Tests

| | LD | MDR | LR |
|---|---|---|---|
| TOTAL | | | |
| GPRO, STAR | X | | |
| LHR3, STAR | X | | |
| LHB4, STAR | X | | |
| LHB5, STAR | X | | |
| LHB3, APOE | X | | |
| FSHR6, FSH2 | X | | |
| FSHR8, FSH2 | X | | |
| APOE, GPRO, FSHR11 | | X | |
| APOE, LHR1, LHR2 | | X | |
| LHB4, FSHR13 | | | X |
| GPRO, FSHR2 | | | X |
| MALES | | | |
| GPRO, FSHR2 | X | | |
| FSHR6, FSH1 | X | | |
| FSHR6, FSH2 | X | | |
| FSHR8, FSH1 | X | | |
| FSHR8, FSH2 | X | | |
| APOE, LHR2 | X | X | X |
| APOE, LHR2, LHR5 | | X | |
| LHR5, LHB2 | | | X |
| LHR5, LHB4 | | | X |
| LHB2, FSHR2 | | | X |
| FEMALES | | | |
| APOE, STAR | X | | |
| LHR2, STAR | X | | |
| LHR4, FSHR11 | X | | |
| LHB1, STAR | X | | |
| LHB4, STAR | X | | |
| LHB5, STAR | X | | |
| FSHR4, STAR | X | | |
| APOE, LHB5 | | X | |
| APOE, FSH2 | | X | |
| ApoE, LHR2, FSH2 | | X | |
| LHR2, FSH1 | | X | X |
| LHR2, FSH2 | | | X |
| LHR4, FSHR11 | | | X |
| LHR4, FSHR13 | | | X |

TABLE 6
Test Numbers and Resulting Alpha Correction

| Dataset | χ² | HWE | Age of Onset | LD | MDR | LR | total | alpha |
|---|---|---|---|---|---|---|---|---|
| total | 62 | 31 | 31 | 465 | 1 | 24 | 614 | 0.0071 |
| male | 62 | 31 | 31 | 465 | 1 | 15 | 605 | 0.0072 |
| female | 62 | 31 | 31 | 465 | 1 | 35 | 625 | 0.0071 |

### REFERENCES

1. Bertram L, Tanzi R E: The current status of Alzheimer's disease genetics: what do we tell the patients? Pharmacol Res 2004, 50(4):385-396.
2. Corder E H, Saunders A M, Strittmatter W J, Schmechel D E, Gaskell P C, Small G W, Roses A D, Haines J L, Pericak-Vance M A: Gene dose of apolipoprotein E type 4 allele and the risk of Alzheimer's disease in late onset families. Science 1993, 261(5123):921-923.
3. Saunders A M, Strittmatter W J, Schmechel D, George-Hyslop P H, Pericak-Vance M A, Joo S H, Rosi B L, Gusella J F, Crapper-MacLachlan D R, Alberts M J et al: Association of apolipoprotein E allele epsilon 4 with late-onset familial and sporadic Alzheimer's disease. Neurology 1993, 43(8):1467-1472.
4. Farrer L A, Cupples L A, Haines J L, Hyman B, Kukull W A, Mayeux R, Myers R H, Pericak-Vance M A, Risch N, van Duijn C M: Effects of age, sex, and ethnicity on the association between apolipoprotein E genotype and Alzheimer disease. A meta-analysis. APOE and Alzheimer Disease Meta Analysis Consortium. Jama 1997, 278(16):1349-1356.
5. Roses A, Devlin B, Conneally P, Small G, Saunders A, Pritchard M: Measuring the genetic contribution of APOE in late-onset Alzheimer disease. Am J Hum Genet 1995, Suppl. 57:A202.
6. Daw E W, Payami H, Nemens E J, Nochlin D, Bird T D, Schellenberg G D, Wijsman E M: The number of trait loci in late-onset Alzheimer disease. Am J Hum Genet 2000, 66(1):196-204.
7. Chapuis J, Tian J, Shi J, Bensemain F, Cottel D, Lendon C, Amouyel P, Mann D, Lambert J C: Association study of the vascular endothelial growth factor gene with the risk of developing Alzheimer's disease. Neurobiol Aging 2006, 27(9):1212-1215.
8. Ozturk A, DeKosky S T, Kamboh M I: Lack of association of 5 SNPs in the vicinity of the insulin-degrading enzyme (IDE) gene with late-onset Alzheimer's disease. Neurosci Lett 2006, 406(3):265-269.
9. Pritchard A, Harris J, Pritchard C W, St Clair D, Lemmon H, Lambert J C, Chartier-Harlin M C, Hayes A, Thaker U, Iwatsubo T et al: Association study and meta-analysis of low-density lipoprotein receptor related protein in Alzheimer's disease. Neurosci Lett 2005, 382(3):221-226.
10. Monastero R, Cefalu A B, Camarda C, Noto D, Camarda L K, Caldarella R, Imbornone E, Averna M R, Camarda R: Association of estrogen receptor alpha gene with Alzheimer's disease: a case-control study. J Alzheimers Dis 2006, 9(3):273-278.
11. Pirskanen M, Hiltunen M, Mannermaa A, Helisalmi S, Lehtovirta M, Hanninen T, Soininen H: Estrogen receptor beta gene variants are associated with increased risk of Alzheimer's disease in women. Eur J Hum Genet 2005, 13(9):1000-1006.
12. Bowen R L, Isley J P, Atkinson R L: An association of elevated serum gonadotropin concentrations and Alzheimer disease? J Neuroendocrinol 2000, 12(4):351-354.
13. Short R A, Bowen R L, O'Brien P C, Graff-Radford N R: Elevated gonadotropin levels in patients with Alzheimer disease. Mayo Clin Proc 2001, 76(9):906-909.
14. Hogervorst E, Bandelow S, Combrinck M, Smith A D: Low free testosterone is an independent risk factor for Alzheimer's disease. Exp Gerontol 2004, 39(11-12):1633-1639.
15. Lukacs H, Hiatt E S, Lei Z M, Rao C V: Peripheral and intracerebroventricular administration of human chorionic gonadotropin alters several hippocampus-associated behaviors in cycling female rats. Horm Behav 1995, 29(1):42-58.
16. Lei Z M, Rao C V: Novel presence of luteinizing hormone/human chorionic gonadotropin (hCG) receptors and the down-regulating action of hCG on gonadotropin-releasing hormone gene expression in immortalized hypothalamic GT1-7 neurons. Mol Endocrinol 1994, 8(8):1111-1121.
17. Bowen R L, Verdile G, Liu T, Parlow A F, Perry G, Smith M A, Martins R N, Atwood C S: Luteinizing hormone, a reproductive regulator that modulates the processing of amyloid-beta precursor protein and amyloid-beta deposition. J Biol Chem 2004, 279(19):20539-20545.
18. Casadesus G, Atwood C S, Bowen R L, Smith M A: Luteinizing hormone modulates cognition and amyloid-beta deposition in Alzheimer APP tansgenic mice. Biochem Biophys Acta 2006.
19. Liu T, Wimalasena J, Bowen R L, Atwood C S: Luteinizing hormone receptor mediates neuronal pregnenolone production via up-regulation of steroidogenic acute regulatory protein expression. J Neurochem 2007, 100(5):1329-1339.
20. Weill-Engerer S, David J P, Sazdovitch V, Liere P, Eychenne B, Pianos A, Schumacher M, Delacourte A, Baulieu E E, Akwa Y: Neurosteroid quantification in human brain regions: comparison between Alzheimer's and nondemented patients. J Clin Endocrinol Metab 2002, 87(11):5138-5143.
21. Simpkins J W, Yang S H, Wen Y, Singh M: Estrogens, progestins, menopause and neurodegeneration: basic and clinical studies. Cell Mol Life Sci 2005, 62(3):271-280.
22. Mahley R W: Apolipoprotein E: cholesterol transport protein with expanding role in cell biology. Science 1988, 240(4852):622-630.
23. Andersen O M, Willnow T E: Lipoprotein receptors in Alzheimer's disease. Trends Neurosci 2006, 29(12):687-694.
24. Themmen APN, Huhtaniemi I T: Mutations of gonadotropins and gonadotropin receptors: elucidating the physiology and pathophysiology of pituitary-gonadal function. Endocr Rev 2000, 21(5):551-583.
25. Huhtaniemi I T, Themmen A P: Mutations in human gonadotropin and gonadotropin-receptor genes. Endocrine 2005, 26(3):207-217.
26. Furui K, Suganuma N, Tsukahara S, Asada Y, Kikkawa F, Tanaka M, Ozawa T, Tomoda Y: Identification of two point mutations in the gene coding luteinizing hormone (LH) beta-subunit, associated with immunologically anomalous LH variants. J Clin Endocrinol Metab 1994, 78(1):107-113.
27. Takahashi K, Kurioka H, Ozaki T, Kanasaki H, Miyazaki K, Karino K: Pituitary response to luteinizing hormone-releasing hormone in women with variant luteinizing hormone. Eur J Endocrinol 2000, 143(3):375-381.
28. Muller T, Gromoll J, Simoni M: Absence of exon 10 of the human luteinizing hormone (LH) receptor impairs LH, but not human chorionic gonadotropin action. J Clin Endocrinol Metab 2003, 88(5):2242-2249.
29. Richter-Unruh A, Martens J W, Verhoef-Post M, Wessels H T, Kors W A, Sinnecker G H, Boehmer A, Drop S L, Toledo S P, Brunner H G et al: Leydig cell hypoplasia: cases with new mutations, new polymorphisms and cases without mutations in the luteinizing hormone receptor gene. Clin Endocrinol (Oxf) 2002, 56(1):103-112.
30. Ashley-Koch A E, Mei H, Jaworski J, Ma D Q, Ritchie M D, Menold M M, Delong G R, Abramson R K, Wright H H, Hussman J P et al: An analysis paradigm for investigating multi-locus effects in complex disease: examination of three GABA receptor subunit genes on 15q11-q13 as risk factors for autistic disorder. Ann Hum Genet 2006, 70(Pt 3):281-292.
31. Ghebremedhin E, Schultz C, Thal D R, Rub U, Ohm T G, Braak E, Braak H: Gender and age modify the association between APOE and AD-related neuropathology. Neurology 2001, 56(12):1696-1701.
32. Payami H, Zareparsi S, Montee K R, Sexton G J, Kaye J A, Bird T D, Yu C E, Wijsman E M, Heston L L, Litt M et al: Gender difference in apolipoprotein E-associated risk for familial Alzheimer disease: a possible clue to the higher incidence of Alzheimer disease in women. Am J Hum Genet 1996, 58(4): 803-811.
33. Lehmann D J, Butler H T, Warden D R, Combrinck M, King E, Nicoll J A, Budge M M, de Jager C A, Hogervorst E, Esiri M M et al: Association of the androgen receptor CAG repeat polymorphism with Alzheimer's disease in men. Neurosci Lett 2003, 340(2):87-90.
34. Smith M A, Perry G, Atwood C S, Bowen R L: Estrogen replacement and risk of Alzheimer disease. Jama 2003, 289(9):1100; author reply 1101-1102.
35. Meethal S V, Smith M A, Bowen R L, Atwood C S: The gonadotropin connection in Alzheimer's disease. Endocrine 2005, 26(3):317-326.
36. Strittmatter W J, Weisgraber K H, Huang D Y, Dong L M, Salvesen G S, Pericak-Vance M, Schmechel D, Saunders A M, Goldgaber D, Roses A D: Binding of human apolipoprotein E to synthetic amyloid beta peptide: isoform-specific effects and implications for late-onset Alzheimer disease. Proc Natl Acad Sci USA 1993, 90(17):8098-8102.
37. Fazekas F, Enzinger C, Ropele S, Schmidt H, Schmidt R, Strasser-Fuchs S: The impact of our genes: consequences of the apolipoprotein E polymorphism in Alzheimer disease and multiple sclerosis. J Neurol Sci 2006, 245(1-2):35-39.
38. Zhang G, Curtiss L K, Wade R L, Dyer C A: An apolipoprotein E synthetic peptide selectively modulates the transcription of the gene for rat ovarian theca and interstitial cell P450 17alpha-hydroxylase, C17-20 lyase. J Lipid Res 1998, 39(12):2406-2414.
39. Foster J D, Strauss J F, 3rd, Paavola L G: Cellular events involved in hormonal control of receptor-mediated endocytosis: regulation occurs at multiple sites in the low density lipoprotein pathway, including steps beyond the receptor. Endocrinology 1993, 132(1):337-350.
40. Milne R L, Ribas G, Gonzalez-Neira A, Fagerholm R, Salas A, Gonzalez E, Dopazo J, Nevanlinna H, Robledo M, Benitez J: ERCC4 Associated with Breast Cancer Risk: A Two-Stage Case-Control Study Using High-throughput Genotyping. Cancer Res 2006, 66(19): 9420-9427.
41. Yu Y, Panhuysen C, Kranzler H R, Hesselbrock V, Rounsaville B, Weiss R, Brady K, Farrer L A, Gelernter J: Intronic variants in the dopa decarboxylase (DDC) gene are associated with smoking behavior in European-Americans and African-Americans. Hum Mol Genet 2006, 15(14):2192-2199.
42. Lin S L, Miller J D, Ying S Y: Intronic MicroRNA (miRNA). J Biomed Biotechnol 2006, 2006(4):26818.
43. Wessagowit V, Nalla V K, Rogan P K, McGrath J A: Normal and abnormal mechanisms of gene splicing and relevance to inherited skin diseases. J Dermatol Sci 2005, 40(2):73-84.
44. Matlin A J, Clark F, Smith C W: Understanding alternative splicing: towards a cellular code. Nat Rev Mol Cell Biol 2005, 6(5):386-398.
45. Churbanov A, Rogozin I B, Deogun J S, Ali H: Method of predicting splice sites based on signal interactions. Biol Direct 2006, 1:10.
46. Lewis P, Zaykin D: Computer program for the analysis of allelic data. Version 1.0(d16c). Free program distributed by the authors over the internet. In.; 2007.
47. Minitab: Minitab v 14.0 State College, Pa. In.
48. Lancaster A, Nelson M P, Meyer D, Thomson G, Single R M: PyPop: a software framework for population genomics: analyzing large-scale multi-locus genotype data. Pac Symp Biocomput 2003:514-525.
49. Hahn L W, Ritchie M D, Moore J H: Multifactor dimensionality reduction software for detecting gene-gene and gene-environment interactions. Bioinformatics 2003, 19(3):376-382.
50. Jiang M, Lamminen T, Pakarinen P, Hellman J, Manna P, Herrera R J, Huhtaniemi I: A novel Ala(-3)Thr mutation in the signal peptide of human luteinizing hormone beta-subunit: potentiation of the inositol phosphate signalling pathway and attenuation of the adenylate cyclase pathway by recombinant variant hormone. Mol Hum Reprod 2002, 8(3):201-212.
51. Atger M, Misrahi M, Sar S, Le Flem L, Dessen P, Milgrom E: Structure of the human luteinizing hormone-choriogonadotropin receptor gene: unusual promoter and 5′ non-coding regions. Mol Cell Endocrinol 1995, 111(2):113-123.
52. Kremer H, Martens J W, van Reen M, Verhoef-Post M, Wit J M, Otten B J, Drop S L, Delemarre-van de Waal H A, Pombo-Arias M, De Luca F et al: A limited repertoire of mutations of the luteinizing hormone (LH) receptor gene in familial and sporadic patients with male LH-independent precocious puberty. J Clin Endocrinol Metab 1999, 84(3): 1136-1140.
53. Thornton-Wells T A, Moore J H, Haines J L: Genetics, statistics and human disease: analytical retooling for complexity. Trends Genet 2004, 20(12):640-647.
54. Peduzzi P, Concato J, Kemper E, Holford T R, Feinstein A R: A simulation study of the number of events per variable in logistic regression analysis. J Clin Epidemiol 1996, 49(12):1373-1379.
55. Ritchie M D, Hahn L W, Roodi N, Bailey L R, Dupont W D, Parl F F, Moore J H: Multifactor-dimensionality reduction reveals high-order interactions among estrogen-metabolism genes in sporadic breast cancer. Am J Hum Genet 2001, 69(1):138-147.
56. Moore J H: The ubiquitous nature of epistasis in determining susceptibility to common human diseases. Hum Hered 2003, 56(1-3):73-82.
57. Coffey C S, Hebert P R, Ritchie M D, Krumholz H M, Gaziano J M, Ridker P M, Brown N J, Vaughan D E, Moore J H: An application of conditional logistic regression and multifactor dimensionality reduction for detecting gene-gene interactions on risk of myocardial infarction: the importance of model validation. BMC Bioinformatics 2004, 5:49.
58. Motsinger A A, Ritchie M D: The effect of reduction in cross-validation intervals on the performance of multifactor dimensionality reduction. Genet Epidemiol 2006, 30(6):546-555.
59. Moore J H, Williams S M: New strategies for identifying gene-gene interactions in hypertension. Ann Med 2002, 34(2):88-95.
60. Williams S M, Addy J H, Phillips J A, 3rd, Dai M, Kpodonu J, Afful J, Jackson H, Joseph K, Eason F, Murray M M et al: Combinations of variations in multiple genes are associated with hypertension. Hypertension 2000, 36(1):2-6.
61. Haasl, R J et al., A luteinizing hormone receptor intronic variant is significantly associated with decreased risk of Alzheimer's disease in males carrying an apolipoprotein E ε4 allele. BMC Medical Genetics 2008, 9:37.
62. Manly J J, Merchant C A, Jacobs D M, et al. Endogenous estrogen levels and Alzheimer's disease among postmenopausal women. Neurology 2000 54:833-7.
63. Benjamini, Y., Drai, D., Elmer, G., Kafkafi, N. & Golani, I. Controlling the false discovery rate in behavior genetics research. Behav Brain Res 125, 279-84 (2001).

1.-25. (canceled)

26. A method for detecting alleles in a genomic DNA sample of a patient, the method comprising:
(a) reacting the genomic DNA sample from the patient with a reaction mixture comprising a primer or probe for detecting an Apolipoprotein E4 (APOE4) allele, and detecting the Apolipoprotein E4 (APOE4) allele;
(b) reacting the genomic DNA sample from the patient with a reaction mixture comprising a primer or probe for detecting an allele of the polymorphic position of rs4073366, and detecting the allele of the polymorphic position of rs4073366; and
(c) reacting the genomic DNA sample from the patient with a reaction mixture comprising a primer or probe for detecting an allele of the polymorphic position of rs6169, and detecting the allele of the polymorphic position of rs6169.

27. The method of claim 26, wherein prior to performing steps (a), (b), and (c), the genomic DNA sample is amplified.

28. The method of claim 27, wherein amplifying comprises:
(i) amplifying the genomic DNA with primers that hybridize to the Apolipoprotein E4 (APOE4) allele;
(ii) amplifying the genomic DNA with primers that hybridize to the allele of the polymorphic position of rs4073366; and
(iii) amplifying the genomic DNA with primers that hybridize to the allele of the polymorphic position of rs6169.

29. The method of claim 26, wherein:
the primer or probe of step (a) specifically hybridizes to the Apolipoprotein E4 (APOE4) allele;
the primer or probe of step (b) specifically hybridizes to the polymorphic position of rs4073366; and
the primer or probe of step (c) specifically hybridizes to the allele of the polymorphic position of rs6169.

30. The method of claim 26, wherein:
the primer or probe of step (a) is labeled with a fluorophore, a radiolabel, an enzyme, or a particulate label;
the primer or probe of step (b) is labeled with a fluorophore, a radiolabel, an enzyme, or a particulate label; and
the primer or probe of step (c) is labeled with a fluorophore, a radiolabel, an enzyme, or a particulate label.

31. The method of claim 26, wherein:
(i) the reaction mixture of step (a) is a reaction mixture for sequencing the Apolipoprotein E4 (APOE4) allele comprising a primer for sequencing the Apolipoprotein E4 (APOE4) allele, and detecting the Apolipoprotein E4 (APOE4) allele comprises sequencing the Apolipoprotein E4 (APOE4) allele;
(ii) the reaction mixture of step (b) is a reaction mixture for sequencing the allele of the polymorphic position of rs4073366 comprising a primer for sequencing the allele of the polymorphic position of rs4073366, and detecting the allele of the polymorphic position of rs4073366 comprises sequencing the allele of the polymorphic position of rs4073366; and
(iii) the reaction mixture of step (c) is a reaction mixture for sequencing the allele of the polymorphic position of rs6169 comprising a primer for sequencing the allele of the polymorphic position of rs6169, and detecting the allele of the polymorphic position of rs6169 comprises sequencing the allele of the polymorphic position of rs6169.

32. The method of claim 26, wherein the reaction mixture of (a), the reaction mixture of (b), and the reaction mixture of (c) are the same reaction mixture.

33. The method of claim 26, wherein:
(i) the reaction mixture of step (a) is a reaction mixture for probing for the Apolipoprotein E4 (APOE4) allele comprising a probe that hybridizes to the Apolipoprotein E4 (APOE4) allele, and detecting the Apolipoprotein E4 (APOE4) allele comprises detecting hybridization of the probe to the Apolipoprotein E4 (APOE4) allele;
(ii) the reaction mixture of step (b) is a reaction mixture for probing for allele of the polymorphic position of rs4073366 comprising a probe that hybridizes to the allele of the polymorphic position of rs4073366, and detecting the allele of the polymorphic position of rs4073366 comprises detecting hybridization of the probe to the allele of the polymorphic position of rs4073366; and
(iii) the reaction mixture of step (c) is a reaction mixture for probing for allele of the polymorphic position of rs6169 comprising a probe that hybridizes to the allele of the polymorphic position of rs6169, and detecting the allele of the polymorphic position of rs6169 comprises detecting hybridization of the probe to the allele of the polymorphic position of rs6169.

34. The method of claim 33, wherein the reaction mixture of step (a), the reaction mixture of step (b), and the reaction mixture of step (c) are the same reaction mixture.

35. A method for detecting alleles in a genomic DNA sample of a patient, the method comprising:
(a) contacting the genomic DNA sample from the patient with an array for detecting an Apolipoprotein E4 (APOE4) allele and detecting the Apolipoprotein E4 (APOE4) allele;
(b) contacting the genomic DNA sample from the patient with an array for detecting an allele of the polymorphic position of rs4073366 and detecting the allele of the polymorphic position of rs4073366; and
(c) contacting the genomic DNA sample from the patient with array for detecting an allele of the polymorphic position of rs6169 and detecting the allele of the polymorphic position of rs6169.

36. The method of claim 35, wherein:
(i) the array of step (a) comprises an immobilized oligonucleotide that hybridizes to the Apolipoprotein E4 (APOE4) allele, and detecting the Apolipoprotein E4 (APOE4) allele comprises hybridizing the Apolipoprotein E4 (APOE4) allele to the immobilized oligonucleotide and amplifying the Apolipoprotein E4 (APOE4) allele to form an amplicon after the Apolipoprotein E4 (APOE4) allele has hybridized to the immobilized oligonucleotide, and detecting the amplicon;
(ii) the array of step (b) comprises an immobilized oligonucleotide that hybridizes to the allele of the polymorphic position of rs4073366, and detecting the allele of the polymorphic position of rs4073366 comprises hybridizing the allele of the polymorphic position of rs4073366 and amplifying the allele of the polymorphic position of rs4073366 to form an amplicon after the allele of the polymorphic position of rs4073366 has hybridized to the immobilized oligonucleotide, and detecting the amplicon; and
(iii) the array of step (c) comprises an immobilized oligonucleotide that hybridizes to the allele of the polymorphic position of rs6169, and detecting the allele of the polymorphic position of rs6169 comprises hybridizing the allele of the polymorphic position of rs46169 and amplifying the allele of the polymorphic position of rs46169 to form an amplicon after the allele of the polymorphic position of rs6169 has hybridized to the immobilized oligonucleotide, and detecting the amplicon.

37. The method of claim 35, wherein the array of step (a), the array of step (b), and the array of step (c) are the same array.

38. A method for detecting alleles in a genomic DNA sample of a patient, the method comprising amplifying the genomic DNA sample and
(a) contacting the amplified genomic DNA sample from the patient with an array for detecting an Apolipoprotein E4 (APOE4) allele and detecting the Apolipoprotein E4 (APOE4) allele;
(b) contacting the amplified genomic DNA sample from the patient with an array for detecting an allele of the polymorphic position of rs4073366 and detecting the allele of the polymorphic position of rs4073366; and
(c) contacting the amplified genomic DNA sample from the patient with array for detecting an allele of the polymorphic position of rs6169 and detecting the allele of the polymorphic position of rs6169.

39. The method of claim 38, wherein:
(i) the array of step (a) comprises an immobilized oligonucleotide that hybridizes to the Apolipoprotein E4 (APOE4) allele;
(ii) the array of step (b) comprises an immobilized oligonucleotide that hybridizes to the allele of the polymorphic position of rs4073366; and
(iii) the array of step (c) comprises an immobilized oligonucleotide that hybridizes to the allele of the polymorphic position of rs6169.

40. The method of claim 38, wherein amplifying the genomic DNA sample comprises:
(i) amplifying the genomic DNA with primers that hybridize to the Apolipoprotein E4 (APOE4) allele;
(ii) amplifying the genomic DNA with primers that hybridize to the allele of the polymorphic position of rs4073366; and
(iii) amplifying the genomic DNA with primers that hybridize to the allele of the polymorphic position of rs6169.

41. The method of claim 38, wherein the array of step (a), the array of step (b), and the array of step (c) are the same array.
